(19) United States
(12) Patent Application Publication
(10) Pub. No.: US 2025/0137061 A1
(43) Pub. Date: May 1, 2025

(54) METHODS FOR DETECTION AND QUANTITATION OF CIRCULATING TUMOR DNA

(71) Applicant: Myriad Women's Health, Inc., South San Francisco, CA (US)

(72) Inventors: Ashley Acevedo, South San Francisco, CA (US); Matthew LaBella, South San Francisco, CA (US); Dale Muzzey, South San Francisco, CA (US)

(73) Assignee: Myriad Women's Health, Inc., South San Francisco, CA (US)

(21) Appl. No.: 18/930,324

(22) Filed: Oct. 29, 2024

Related U.S. Application Data
(60) Provisional application No. 63/546,464, filed on Oct. 30, 2023.

(51) Int. Cl. C12Q 1/6886; C12Q 1/6874

(52) U.S. Cl. CPC C12Q 1/6886; C12Q 1/6874

(57) ABSTRACT

Provided herein is a method of detecting circulating tumor DNA (ctDNA) in a sample. The method utilizes a preliminary panel that is cross-referenced with a second non-tumor sample to detect the presence or absence of any of the plurality of preliminary tumor-specific somatic mutations and removing said plurality of preliminary tumor-specific somatic mutations to obtain a patient-specific panel that can be used to detect ctDNA in further samples.

### CROSS REFERENCE TO RELATED APPLICATION

This application claims the benefit under 35 U.S.C. § 119(e) of U.S. Provisional Application No. 63/546,464, filed Oct. 30, 2023, the contents of which are incorporated herein by reference in their entirety.

### TECHNICAL FIELD

Described herein are methods of improving the sensitivity and specificity of tumor-informed minimal residual disease (MRD) assays and panels. More specifically, the present disclosure provides methods for generating more specific patient-specific panels for use in MRD assays.

### BACKGROUND

The following description of the background of the present technology is provided simply as an aid in understanding the present technology and is not admitted to describe or constitute prior art to the present technology.

The discovery of cell-free deoxyribonucleic acid (cfDNA) has promoted the non-invasive detection of alternations in genomic sequences that occur in various disease states. However, in some instances, e.g., cancer, the ability to determine the presence of disease by detecting disease-associated mutations has been hindered by the extremely low levels of cell-free tumor DNA. Methods that allow for the accurate detection of disease-associated mutations remain desirable. In addition, there also remains a need for the determination of tumor fraction in pre- and post-treatment cancer patients and ensuring that the methods used have reduced error and false positive rates.

### SUMMARY

The present disclosure provides methods of detecting and/or quantitating circulating tumor DNA by incorporating additional quality control steps to remove any mutations that are incorrectly associated with a patient's specific cancer.

In a first aspect, the present disclosure provides methods of generating a patient-specific panel, comprising: (a) obtaining from a tumor sample and a first non-tumor sample from a subject that has been diagnosed with a cancer; (b) sequencing DNA from the tumor sample and the first non-tumor sample; (c) preparing a preliminary panel comprising a plurality of preliminary tumor-specific somatic mutations that are present in the tumor sample and absent in the first non-tumor sample; (d) obtaining a second non-tumor sample from the subject; (c) extracting DNA from the second non-tumor sample; (f) enriching the DNA from the second non-tumor sample for sequences corresponding to the preliminary panel, thereby obtaining an enriched DNA fraction from the second non-tumor sample; (g) sequencing the enriched DNA fraction from the second non-tumor sample to detect the presence or absence of any of the plurality of preliminary tumor-specific somatic mutations; and (h) removing from the preliminary panel any of the plurality of preliminary tumor-specific somatic mutations that are present in the enriched DNA fraction from the second non-tumor sample, thereby generating a patient-specific panel. In some embodiments, the method may further comprise preparing a plurality of oligonucleotide probes, wherein each probe in the plurality of oligonucleotide probes hybridizes to a tumor-specific somatic mutation in the patient-specific signature panel.

In a second aspect, the present disclosure provides methods of detecting circulating tumor DNA (ctDNA) in a sample, comprising: (a) obtaining from a tumor sample and a first non-tumor sample from a subject that has been diagnosed with a cancer; (b) sequencing DNA from the tumor sample and the first non-tumor sample; (c) preparing a preliminary panel comprising a plurality of preliminary tumor-specific somatic mutations that are present in the tumor sample and absent in the first non-tumor sample; (d) obtaining a second non-tumor sample from the subject; (c) extracting DNA from the second non-tumor sample; (f) enriching the DNA from the second non-tumor sample for sequences corresponding to the preliminary panel, thereby obtaining an enriched DNA fraction from the second non-tumor sample; (g) sequencing the enriched DNA fraction from the second non-tumor sample to detect the presence or absence of any of the plurality of preliminary tumor-specific somatic mutations; (h) removing from the preliminary panel any of the plurality of preliminary tumor-specific somatic mutations that are present in the enriched DNA fraction from the second non-tumor sample, thereby obtaining a patient-specific panel; and at one or more later time points: (i) obtaining a third non-tumor sample from the subject; (j) extracting cfDNA from the third non-tumor sample; (k) enriching the cfDNA from the third non-tumor sample for sequences corresponding to the patient-specific panel, thereby obtaining an enriched DNA fraction from the third non-tumor sample; and (l) sequencing the enriched DNA fraction from the third non-tumor sample to detect the presence or absence of ctDNA in the third non-tumor sample.

In a third aspect, the present disclosure provides methods of detecting minimal residual disease in a subject previously diagnosed with cancer, comprising: (a) obtaining from a tumor sample and a first non-tumor sample from the subject; (b) sequencing DNA from the tumor sample and the first non-tumor sample; (c) preparing a preliminary panel comprising a plurality of preliminary tumor-specific somatic mutations that are present in the tumor sample and absent in the first non-tumor sample; (d) obtaining a second non-tumor sample from the subject; (c) extracting DNA from the second non-tumor sample; (f) enriching the DNA from the second non-tumor sample for sequences corresponding to the preliminary panel, thereby obtaining an enriched DNA fraction from the second non-tumor sample; (g) sequencing the enriched DNA fraction from the second non-tumor sample to detect the presence or absence of any of the plurality of preliminary tumor-specific somatic mutations; (h) removing from the preliminary panel any of the plurality of preliminary tumor-specific somatic mutations that are present in the enriched DNA fraction from the second non-tumor sample, thereby obtaining a patient-specific panel; and at one or more later time points: (i) obtaining a third non-tumor sample from the subject; (j) extracting cfDNA from the third non-tumor sample; (k) enriching the cfDNA from the third non-tumor sample for sequences corresponding to the patient-specific panel, thereby obtaining an enriched DNA fraction from the third non-tumor sample; and (l) sequencing the enriched DNA fraction from the third non-tumor sample to detect the presence or absence of ctDNA in the third non-tumor sample, wherein the presence of ctDNA in the third non-tumor sample is indicative of minimal residual disease in the subject.

In a fourth aspect, the present disclosure provides methods of monitoring treatment in a subject with cancer, comprising: (a) obtaining from a tumor sample and a first non-tumor sample from the subject; (b) sequencing DNA from the tumor sample and the first non-tumor sample; (c) preparing a preliminary panel comprising a plurality of preliminary tumor-specific somatic mutations that are present in the tumor sample and absent in the first non-tumor sample; (d) obtaining a second non-tumor sample from the subject; (e) extracting DNA from the second non-tumor sample; (f) enriching the DNA from the second non-tumor sample for sequences corresponding to the preliminary panel, thereby obtaining an enriched DNA fraction from the second non-tumor sample; (g) sequencing the enriched DNA fraction from the second non-tumor sample to detect the presence or absence of any of the plurality of preliminary tumor-specific somatic mutations; (h) removing from the preliminary panel any of the plurality of preliminary tumor-specific somatic mutations that are present in the enriched DNA fraction from the second non-tumor sample, thereby obtaining a patient-specific panel; and at one or more later time points: (i) obtaining a third non-tumor sample from the subject; (j) extracting cfDNA from the third non-tumor sample; (k) enriching the cfDNA from the third non-tumor sample for sequences corresponding to the patient-specific panel, thereby obtaining an enriched DNA fraction from the third non-tumor sample; and (l) sequencing the enriched DNA fraction from the third non-tumor sample to detect the presence or absence of ctDNA in the third non-tumor sample, wherein the presence or absence of ctDNA in the third non-tumor sample is indicative of the effectiveness of a treatment.

In a fifth aspect, the present disclosure provides methods of monitoring cancer recurrence or disease progression in a subject previously treated for cancer, comprising: (a) obtaining from a tumor sample and a first non-tumor sample from the subject; (b) sequencing DNA from the tumor sample and the first non-tumor sample; (c) preparing a preliminary panel comprising a plurality of preliminary tumor-specific somatic mutations that are present in the tumor sample and absent in the first non-tumor sample; (d) obtaining a second non-tumor sample from the subject; (c) extracting DNA from the second non-tumor sample; (f) enriching the DNA from the second non-tumor sample for sequences corresponding to the preliminary panel, thereby obtaining an enriched DNA fraction from the second non-tumor sample; (g) sequencing the enriched DNA fraction from the second non-tumor sample to detect the presence or absence of any of the plurality of preliminary tumor-specific somatic mutations; (h) removing from the preliminary panel any of the plurality of preliminary tumor-specific somatic mutations that are present in the enriched DNA fraction from the second non-tumor sample, thereby obtaining a patient-specific panel; and at one or more later time points: (i) obtaining a third non-tumor sample from the subject; (j) extracting cfDNA from the third non-tumor sample; (k) enriching the cfDNA from the third non-tumor sample for sequences corresponding to the patient-specific panel, thereby obtaining an enriched DNA fraction from the third non-tumor sample; and (l) sequencing the enriched DNA fraction from the third non-tumor sample to detect the presence or absence of ctDNA in the third non-tumor sample, wherein the presence of ctDNA in the third non-tumor sample is indicative of recurrence or progression of disease.

For the purposes of any of the foregoing aspects, the first non-tumor sample and the second non-tumor sample may be the same sample. For example, the first non-tumor sample may be a buffy coat sample obtained by a blood draw from the patient. The same buffy coat sample may be saved and used as the second non-tumor sample to screen the preliminary panel. Alternatively, the second non-tumor sample could be a different non-tumor sample, such as a subsequent buffy coat sample obtained from a separate blood draw.

In some embodiments of any of the forgoing aspects, enriching the DNA from the second non-tumor sample comprises contacting the DNA from the second non-tumor sample with a plurality of oligonucleotides, wherein each oligonucleotide in the plurality of oligonucleotides comprises a nucleic acid sequence that is capable of hybridizing to a DNA fragment comprising one of the plurality of preliminary tumor-specific somatic mutations. In some embodiments of any of the forgoing aspects, enriching the DNA from the second non-tumor sample comprises (i) hybrid capture-based enrichment, (ii) PCR-target enrichment, or (iii) on-sequencer enrichment.

In some embodiments of any of the second through fifth aspects, enriching the cfDNA from the third non-tumor sample comprises contacting the cfDNA from the third non-tumor sample with a patient-specific plurality of oligonucleotides, wherein each oligonucleotide in the patient-specific plurality of oligonucleotides comprises a nucleic acid sequence that is capable of hybridizing to a DNA fragment comprising a tumor-specific somatic mutation in the patient-specific panel. In some embodiments of any of the second through fifth aspects, wherein enriching the cfDNA from the third non-tumor sample comprises (i) hybrid capture-based enrichment, (ii) PCR-target enrichment, or (iii) on-sequencer enrichment.

In some embodiments of any of the forgoing aspects or embodiments, preparing the preliminary panel comprises aligning the sequences of DNA from the tumor sample to a reference human genome that is not from the patient, aligning the sequences of DNA from the first non-tumor sample to the reference genome that is not from the patient, and detecting the presence of mutations that are present in the sequences of DNA from the tumor sample but absent in the sequences of DNA from the first non-tumor sample.

In some embodiments of any of the second through fifth aspects, the methods may further comprise providing the subject with a report providing a result to the sequencing of the enriched DNA fraction from the third non-tumor sample.

In some embodiments of any of the forgoing aspects or embodiments, sequencing DNA from the tumor sample and the first non-tumor sample comprises whole genome sequencing or targeted sequencing. In some embodiments, the targeted sequencing comprises sequencing of introns, exons, intergenic regions, or a combination thereof.

In some embodiments of any of the forgoing aspects or embodiments, the patient-specific panel comprises at least 10, at least 50, at least 100, at least 150, at least 200, at least 250, at least 500, at least 750, at least 1000, at least 1100, at least 1200, at least 1300, at least 1400, at least 1500, at least 1600, at least 1700, at least 1800, at least 1900, or at least 2000 tumor-specific somatic mutations.

In some embodiments of any of the forgoing aspects or embodiments, the patient-specific panel comprises one or more somatic mutations selected from SNVs, insertions, deletions, and translocations.

In some embodiments of any of the forgoing aspects or embodiments, any of the plurality of preliminary tumor-specific somatic mutations that are removed from the preliminary panel are alterations selected from germ-line changes, clonal hematopoiesis of indeterminate potential (CHIP) mutations, sequencing errors, and artifacts.

In some embodiments of any of the second through fifth aspects, the methods may further comprise determining a tumor fraction. In some embodiments, a tumor fraction of zero indicates the absence of the tumor in the subject.

In some embodiments of any of the forgoing aspects, the tumor sample comprises a solid tumor biopsy or a fluid sample. In some embodiments, the fluid sample is selected from blood, blood plasma, blood serum, urine, saliva, and cerebral spinal fluid (CSF).

In some embodiments of any of the forgoing aspects or embodiments, the first non-tumor sample comprises a tissue sample matched to a tissue of origin of the tumor sample.

In some embodiments of any of the forgoing aspects or embodiments, the first non-tumor sample comprises a fluid sample selected from a buffy coat sample, blood, blood plasma, blood serum, urine, saliva, and cerebral spinal fluid (CSF).

In some embodiments of any of the forgoing aspects or embodiments, the second non-tumor sample comprises a tissue sample matched to a tissue of origin of the tumor sample.

In some embodiments of any of the forgoing aspects or embodiments, the second non-tumor sample comprises a fluid sample selected from a buffy coat sample, blood, blood plasma, blood serum, urine, saliva, and cerebral spinal fluid (CSF).

In some embodiments of any of the second through fifth aspects, the third non-tumor sample comprises a tissue sample matched to a tissue of origin of the tumor sample.

In some embodiments of any of the second through fifth aspects, the third non-tumor sample comprises a fluid sample selected from a buffy coat sample, blood, blood plasma, blood serum, urine, saliva, and cerebral spinal fluid (CSF).

In some embodiments of any of the forgoing aspects or embodiments, the subject has completed at least one cancer treatment prior to obtaining the tumor sample and the first and second non-tumor sample from the subject.

In some embodiments of any of the second through fifth aspects, the subject has completed at least one cancer treatment prior to obtaining the third non-tumor sample from the subject.

In some embodiments of any of the second through fifth aspects, the subject is receiving at least one cancer treatment at the time of obtaining the third non-tumor sample from the subject. In some embodiments, the cancer treatment is selected from chemotherapy, radiotherapy, surgery, immunotherapy, cell therapy, or biologic therapy.

In some embodiments of any of the second through fifth aspects, the methods may further comprise repeating (i)-(l) with a fourth, fifth, sixth, seventh, eighth, ninth, or tenth non-tumor sample at successive time points. In some embodiments, (i)-(l) are repeated one or more times while the subject is in remission. In some embodiments, (i)-(l) are repeated one or more times while the subject is undergoing treatment for the cancer. In some embodiments, (i)-(l) are repeated one or more times coinciding with or prior to surgery; following, during, or prior to administration of chemotherapy; following, during, or prior to radiation therapy; following, during, or prior to administration of an immunotherapy; following, during, or prior to administration of a cell therapy; or following, during, or prior to administration of a biologic therapy.

In some embodiments of any of the forgoing aspects or embodiments, the tumor is selected from adrenal cancer, anal cancer, bile duct cancer, bladder cancer, bone cancer, a brain/CNS tumor, breast cancer, Castleman disease, cervical cancer, colon or rectum cancer, endometrial cancer, esophagus cancer, a Ewing tumor, eye cancer, gallbladder cancer, a gastrointestinal carcinoid tumor, a gastrointestinal stromal tumor (GIST), gestational trophoblastic disease, Hodgkin disease, Kaposi sarcoma, kidney cancer, laryngeal and hypopharyngeal cancer, leukemia, liver cancer, lung cancer, lymphoma, malignant mesothelioma, multiple myeloma, myelodysplastic Syndrome, nasal cavity or paranasal sinus cancer, nasopharyngeal cancer, neuroblastoma, oral cavity or oropharyngeal cancer, osteosarcoma, ovarian cancer, pancreatic cancer, penile cancer, a pituitary tumor, prostate cancer, retinoblastoma, rhabdomyosarcoma, salivary gland cancer, skin cancer, small intestine cancer, stomach cancer, testicular cancer, thymus cancer, thyroid cancer, uterine sarcoma, vaginal cancer, vulvar cancer, Waldenstrom macroglobulinemia, and Wilms tumor.

### BRIEF DESCRIPTION OF THE DRAWINGS

FIG. 1 shows an exemplary workflow for the methods of detecting and quantifying circulating tumor DNA.

FIG. 2 shows the allele frequent of somatic targets identified in a normal control sample.

FIG. 3 depicts the allele frequency of somatic targets detected in 1% and 0.1% test tumor fraction samples.

FIG. 4 shows the tumor fraction (TF) estimates for panels containing or not containing targets detected in a normal control, also termed “false targets.”

FIG. 5 depicts the estimated tumor fraction for panels with false targets removed.

### DETAILED DESCRIPTION

The present disclosure provides a method for detecting and quantifying circulating tumor DNA in a sample from a subject. The present disclosure provides a method of detecting circulating tumor DNA (ctDNA) by generating a panel based on the comparison of tumor and non-tumor cells and later comparing a sample from the subject to the panel to detect the presence or absence of ctDNA in the final sample. The present disclosure also provides an integrated quality control step to remove any non-disease associated mutations from the final patient-specific panel utilized in the MRD assay.

It is to be appreciated that certain aspects, modes, embodiments, variations and features of the present methods are described below in various levels of detail in order to provide a substantial understanding of the present technology. It is to be understood that the present disclosure is not limited to particular uses, methods, reagents, compounds, compositions or biological systems, which can, of course, vary. It is also to be understood that the terminology used herein for the purpose of describing particular embodiments only and is not intended to be limiting.

### I. Definitions

As used herein, the singular forms “a,” “an,” and “the” include the plural reference unless the context clearly dictates otherwise.

Reference to “about” a value or parameter herein includes (and describes) variations that are directed to that value or parameter per se. The term “about” is used herein to mean plus or minus ten percent (10%) of a value. For example, “about 100” refers to any number between 90 and 110.

It is understood that aspects and variations of the invention described herein include “consisting” and/or “consisting essentially of” aspects and variations.

A “set” of reads refers to all sequencing reads with a common parent nucleic acid strand, which may or may not have had errors introduced during sequencing or amplification of the parent nucleic acid strand.

Numeric ranges are inclusive of the numbers defining the range.

The term “mutation” herein refers to a change introduced into a reference sequence, including, but not limited to, substitutions, insertions, deletions (including truncations) relative to the reference sequence. Mutations can involve large sections of DNA (e.g., copy number variation). Mutations can involve whole chromosomes (e.g., aneuploidy). Mutations can involve small sections of DNA. Examples of mutations involving small sections of DNA include, e.g., point mutations or single nucleotide polymorphisms (SNPs), multiple nucleotide polymorphisms, insertions (e.g., insertion of one or more nucleotides at a locus but less than the entire locus), multiple nucleotide changes, deletions (e.g., deletion of one or more nucleotides at a locus), inversions (e.g., reversal of a sequence of one or more nucleotides), an genomic rearrangements (e.g., deletions, duplications, inversions, and translocations). In some embodiments, the reference sequence is a parental sequence. In some embodiments, the reference sequence is a reference human genome, e.g., h19. In some embodiments, the reference sequence is derived from a non-cancer (or non-tumor) sequence. In some embodiments, the mutation is inherited. In some embodiments, the mutation is spontaneous or de nova. In some embodiments, the mutation is a “somatic” mutation or variant.

The term “somatic variant” or “somatic mutation” herein refers to a variant arising after conception, in non-germline DNA of an individual. Somatic variants may include single-nucleotide variants (SNVs) multi-nucleotide variants, insertions and deletions (e.g., indel variants), and genomic rearrangements for example. The terms “somatic variant” and “somatic mutation” are used interchangeably herein.

The term “patient-specific panel” herein refers to a collection of sequences comprising somatic mutations that are specific to a patient, or markers that distinguish between two or more individuals. A panel may distinguish one sample from another.

The term “subset panel” herein refers to a subset of somatic variants of the patient-specific panel. A subset panel may comprise one or more particular types of somatic variants. For example, a subset panel of the patient specific panel may comprise one or more of SNVs multi-nucleotide variants, insertions and deletions, and genomic rearrangements. In some embodiments, the subset panel does not contain SNVs.

The term “tumor burden” herein refers to the total amount of tumor material present in a patient, which can be reflected by the tumor fraction as determined according to the methods provided herein.

The term “tumor fraction” herein refers to the proportion of circulating cell-free tumor DNA (ctDNA) relative to the total amount of cell-free DNA (cfDNA). Tumor fraction may be indicative of the size of the tumor.

The term “genomic DNA” refers to DNA of a cellular genome. The genomic DNA can be cellular, i.e., contained within a cell, or it can be cell free.

The term “sample” herein refers to any substance containing or presumed to contain nucleic acid. The sample can be a biological sample obtained from a subject. The nucleic acids can be RNA, DNA, e.g., genomic DNA. In some embodiments, the biological sample is a biological fluid sample. The fluid sample can be whole blood, plasma, serum, ascites, cerebrospinal fluid, sweat, urine, tears, saliva, buccal sample, cavity rinse, or organ rinse. The fluid sample can be an essentially cell-free liquid sample (e.g., plasma, serum, sweat, urine, tears, etc.). In other embodiments, the biological sample is a solid biological sample, e.g., feces or tissue biopsy, such as a tumor biopsy. In some embodiments, the sample is blood, plasma, or serum.

The term “target sequence” herein refers to a selected target polynucleotide, e.g., a sequence present in a cfDNA molecule or ctDNA molecule, whose presence, amount, and/or nucleotide sequence, or changes in these, are desired to be determined. Target sequences are interrogated for the presence or absence of a somatic variant. The target polynucleotide can be a region of gene associated with a disease. In some embodiments, the region is an exon. The disease can be cancer.

The terms “anneal,” “hybridize,” or “bind,” can refer to two polynucleotide sequences, segments or strands, and can be used interchangeably and have the usual meaning in the art. Two complementary sequences (e.g., DNA and/or RNA) can anneal or hybridize by forming hydrogen bonds with complementary bases to produce a double-stranded polynucleotide or a double-stranded region of a polynucleotide.

The term “marker” or “segregating marker” refers to a moiety that is used to discriminate between two or more samples, e.g., two or more individuals or tissues. A marker may be a nucleic acid (e.g., a gene), small molecule, peptide, fatty acid, metabolite, protein, lipid, etc. A marker may be a mutation. A marker may be a synthetic nucleic acid. A marker or set of markers may define a genetic signature of an entity, e.g., an individual, relative to a second nucleic acid, e.g., a reference nucleic acid sequence.

The terms “treat,” “treatment,” and “treating” refer to the reduction or amelioration of the progression, severity, and/or duration of a proliferative disorder e.g., cancer, or the amelioration of a proliferative disorder resulting from the administration of one or more therapies.

As used herein, the term “barcode” (also termed single molecule identifier or SMI) refers to a known nucleic acid sequence that allows some feature of a polynucleotide with which the barcode is associated to be identified. In some embodiments, the feature of the polynucleotide to be identified is the sample from which the polynucleotide is derived. In some embodiments, barcodes are about or at least about 3, 4, 5, 6, 7, 8, 9, 10, 11, 12, 13, 14, 15, or more nucleotides in length. In some embodiments, barcodes are shorter than 10, 9, 8, 7, 6, 5, or 4 nucleotides in length. In some embodiments, barcodes associated with some polynucleotides are of different lengths than barcodes associated with other polynucleotides. In general, barcodes are of sufficient length and include sequences that are sufficiently different to allow the identification of samples based on barcodes with which they are associated. In some embodiments, a barcode, and the sample source with which it is associated, can be identified accurately after the mutation, insertion, or deletion of one or more nucleotides in the barcode sequence, such as the mutation, insertion, or deletion of 1, 2, 3, 4, 5, 6, 7, 8, 9, 10, or more nucleotides. In some embodiments, each barcode in a plurality of barcodes differ from every other barcode in the plurality at least three nucleotide positions, such as at least 3, 4, 5, 6, 7, 8, 9, 10, or more nucleotide positions. A plurality of barcodes may be represented in a pool of samples, each sample including polynucleotides comprising one or more barcodes that differ from the barcodes contained in the polynucleotides derived from the other samples in the pool. Samples of polynucleotides including one or more barcodes can be pooled based on the barcode sequences to which they are joined, such that all four of the nucleotide bases A, G, C, and T are approximately evenly represented at one or more positions along each barcode in the pool (such as at 1, 2, 3, 4, 5, 6, 7, 8, or more positions, or all positions of the barcode).

The term “copy number variant” or “CNV” refers to any duplication or deletion of a genomic segment.

The term “small nucleotide polymorphism” or “SNP” refers to a single-nucleotide variant (SNV), a multi-nucleotide variant (MNV), or an indel variant about 100 base pairs or less.

The term “multi-nucleotide variant” or “MNV” herein refers to a variant having 2 or more adjacent nucleotide changes.

The term “derived from” encompasses the terms “originated from,” “obtained from,” “obtainable from,” “isolated from,” and “created from,” and generally indicates that one specified material (e.g., a biological sample) finds its origin in another specified material or individual or has features that can be described with reference to another specified material.

The term “library” herein refers to a collection or plurality of template molecules, i.e., target DNA duplexes, which share common sequences at their 5′ ends and common sequences at their 3′ ends. Use of the term “library” to refer to a collection or plurality of template molecules should not be taken to imply that the templates making up the library are derived from a particular source, or that the “library” has a particular composition. By way of example, use of the term “library” should not be taken to imply that the individual templates within the library must be of different nucleotide sequence or that the templates must be related in terms of sequence and/or source.

The term “Next Generation Sequencing” or “NGS” refers to sequencing methods that allow for massively parallel sequencing of clonally amplified and of single nucleic acid molecules during which a plurality, e.g., millions, of nucleic acid fragments from a single sample or from multiple different samples are sequenced in unison. Non-limiting examples of NGS include sequencing-by-synthesis, sequencing-by-ligation, real-time sequencing, and nanopore sequencing.

The term “sequence read” or simply “read” herein refers to sequence information of a nucleic acid fragment obtained through a sequencing assay, such as a next generation sequencing (NGS) assay. In some embodiments, a sequence read refers to data representing a sequence of nucleotide bases that were measured using a clonal sequencing method. Clonal sequencing may produce sequence data representing single, or clones, or clusters of one original DNA molecule. A sequence read may also have associated quality score at each base position of the sequence indicating the probability that nucleotide has been called correctly.

The term “mapping a sequence read” herein refers to the process of determining a sequence read's location of origin in the genome sequence of a particular organism. The location of origin of sequence reads is based on similarity of nucleotide sequence of the read and the genome sequence.

The term “preferential enrichment” of DNA that corresponds to a locus, or preferential enrichment of DNA at a locus, refers to any method that results in the percentage of molecules of DNA in a post-enrichment DNA mixture that correspond to the locus being higher than the percentage of molecules of DNA in the pre-enrichment DNA mixture that correspond to the locus. The method may involve selective amplification of DNA molecules that correspond to a locus. The method may involve removing DNA molecules that do not correspond to the locus. The method may involve a combination of methods. The degree of enrichment is defined as the percentage of molecules of DNA in the post-enrichment mixture that correspond to the locus divided by the percentage of molecules of DNA in the pre-enrichment mixture that correspond to the locus. Preferential enrichment may be carried out at a plurality of loci. In some embodiments of the present disclosure, the degree of enrichment is greater than 20. In some embodiments of the present disclosure, the degree of enrichment is greater than 200. In some embodiments of the present disclosure, the degree of enrichment is greater than 2,000. When preferential enrichment is carried out at a plurality of loci, the degree of enrichment may refer to the average degree of enrichment of all of the loci in the set of loci.

The term “amplification,” with respect to nucleic acid sequences, herein refers to methods that increase the representation of a population of nucleic acid sequences in a sample. Copies of a particular target nucleic acid sequence generated in vitro in an amplification reaction are called “amplicons” or “amplification products”. Amplification may be exponential or linear. A target nucleic acid may be DNA (such as, for example, genomic DNA, cfDNA, ctDNA, and cDNA) or RNA. While the exemplary methods described hereinafter relate to amplification using polymerase chain reaction (PCR), numerous other methods such as isothermal methods, rolling circle methods, etc., are available to the skilled artisan. The skilled artisan will understand that these other methods may be used either in place of, or together with, PCR methods. See, e.g., Saiki, “Amplification of Genomic DNA” in PCR PROTOCOLS, Innis et al., Eds., Academic Press, San Diego, CA 1990, pp 13-20; Wharam, et al., Nucleic Acids Res. 29(11):E54-E54 (2001).

The term “selective amplification” herein refers to a method that increases the number of copies of a particular molecule of DNA, or molecules of DNA that correspond to a particular region of DNA. It may also refer to a method that increases the number of copies of a particular targeted molecule of DNA, or targeted region of DNA more than it increases non-targeted molecules or regions of DNA. Selective amplification may be a method of preferential enrichment.

The term “direct amplification” herein refers to a nucleic acid amplification reaction in which the target nucleic acid is amplified from the sample without prior purification, extraction, or concentration.

The term “amplification mixture” herein refers to a mixture of reagents that are used in a nucleic acid amplification reaction, but does not contain primers or sample. An amplification mixture comprises a buffer, dNTPs, and a DNA polymerase. An amplification mixture may further comprise at least one of MgCl₂, KCl, nonionic and ionic detergents (including cationic detergents). In general, amplification methods disclosed herein with include an amplification mixture. The term “amplification master mix” refers to an amplification mixture, primers, and/or probes for amplifying one or more target nucleic acids, but does not contain the sample to be amplified. The term “reaction-sample mixture” herein refers to a mixture containing amplification master mix and a sample.

The term “multiplex PCR” herein refers to the simultaneous generation of two or more PCR products or amplicons within the same reaction vessel. Similarly, a “2-plex PCR” refers to the simultaneous generation of two PCR products or amplicons within the same reaction vessel. Each PCR product is primed using a distinct primer pair. A multiplex reaction may further include specific probes for each product that are labeled with different detectable moieties.

The term “universal priming sequence” refers to a DNA sequence that may be appended to a population of target DNA molecules, for example by ligation, PCR, or ligation mediated PCR. Once added to the population of target molecules, primers specific to the universal priming sequences can be used to amplify the target population using a single pair of amplification primers. Universal priming sequences are typically not related to the target sequences.

The term “universal adapters” or “ligation adaptors” or “library tags” are DNA molecules containing a universal priming sequence that can be covalently linked to the 5-prime and 3-prime end of a population of target double stranded DNA molecules. The addition of the adapters provides universal priming sequences to the 5-prime and 3-prime end of the target population from which PCR amplification can take place, amplifying all molecules from the target population, using a single pair of amplification primers.

The term “targeting” herein refers to a method used to selectively amplify or otherwise preferentially enrich those molecules of DNA that correspond to a set of loci, in a mixture of DNA.

The term “primer” herein refers to an oligonucleotide, whether occurring naturally or produced synthetically, which is capable of acting as a point of initiation of nucleic acid synthesis when placed under conditions in which synthesis of a primer extension product which is complementary to a nucleic acid strand is induced, e.g., in the presence of four different nucleotide triphosphates and a polymerase enzyme, e.g., a thermostable enzyme, in an appropriate buffer (“buffer” includes pH, ionic strength, cofactors, etc.) and at a suitable temperature. The primer is preferably single-stranded for maximum efficiency in amplification, but may alternatively be double-stranded. If double-stranded, the primer is first treated to separate its strands before being used to prepare extension products. Preferably, the primer is an oligodeoxyribonucleotide. The primer must be sufficiently long to prime the synthesis of extension products in the presence of the polymerase, e.g., thermostable polymerase enzyme. The exact lengths of a primer will depend on many factors, including temperature, source of primer and use of the method. For example, depending on the complexity of the target sequence, the oligonucleotide primer typically contains 15-25 nucleotides, although it may contain more or few nucleotides. Short primer molecules generally require colder temperatures to form sufficiently stable hybrid complexes with template.

A “hybrid capture probe” herein refers to any nucleic acid sequence, possibly modified, that is generated by various methods such as PCR or direct synthesis and intended to be complementary to one strand of a specific target DNA sequence in a sample. The exogenous hybrid capture probes may be added to a prepared sample and hybridized through a denature-reannealing process to form duplexes of exogenous-endogenous fragments. These duplexes may then be physically separated from the sample by various means.

The term “sequencing library” herein refers to DNA that is processed for sequencing, e.g., using massively parallel methods, e.g., NGS. The DNA may optionally be amplified to obtain a population of multiple copies of processed DNA, which can be sequenced by NGS.

A “spacer” may consist of a repeated single nucleotide (e.g., 1, 2, 3, 4, 5, 6, 7, 8, 9, 10, or more of the same nucleotide in a row), or a sequence of 2, 3, 4, 5, 6, 7, 8, 9, 10, or more nucleotides repeated 1, 2, 3, 4, 5, 6, 7, 8, 9, 10, or more times. A spacer may comprise or consist of a specific sequence, such as a sequence that does not hybridize to any target sequence in a sample. A spacer may comprise or consist of a sequence of randomly selected nucleotides.

The phrases “substantially similar” and “substantially identical” in the context of at least two nucleic acids typically means that a polynucleotide includes a sequence that has at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or even 99.5% sequence identity, in comparison with a reference (e.g., wild-type) polynucleotide or polypeptide. Sequence identity may be determined using known programs such as BLAST, ALIGN, and CLUSTAL using standard parameters. (Sec, e.g., Altshul et al. (1990) J. Mol. Biol. 215:403-410; Henikoff et al. (1989) Proc. Natl. Acad. Sci. 89:10915; Karin et al. (1993) Proc. Natl. Acad. Sci. 90:5873; and Higgins et al. (1988) Gene 73:237). Software for performing BLAST analyses is publicly available through the National Center for Biotechnology Information. Also, databases may be searched using FASTA (Person et al. (1988) Proc. Natl. Acad. Sci. 85:2444-2448.) In some embodiments, substantially identical nucleic acid molecules hybridize to each other under stringent conditions (e.g., within a range of medium to high stringency).

The term “tag” refers to a detectable moiety that may be one or more atom(s) or molecule(s), or a collection of atoms and molecules. A tag may provide an optical, fluorescent, electrochemical, magnetic, or electrostatic (e.g., inductive, capacitive) signature.

The term “tagged nucleotide” herein refers to a nucleotide that includes a tag (or tag species) that is coupled to any location of the nucleotide including, but not limited to a phosphate (e.g., terminal phosphate), sugar or nitrogenous base moiety of the nucleotide. Tags may be one or more atom(s) or molecule(s), or a collection of atoms and molecules. A tag may provide an optical, electrochemical, magnetic, or electrostatic (e.g., inductive, capacitive) signature.

As used herein, the term “target polynucleotide” refers to a nucleic acid molecule or polynucleotide in a population of nucleic acid molecules having a target sequence to which one or more oligonucleotides are designed to hybridize. “Target polynucleotide” may be used to refer to a double-stranded nucleic acid molecule that includes a target sequence on one or both strands, or a single-stranded nucleic acid molecule including a target sequence, and may be derived from any source of or process for isolating or generating nucleic acid molecules. A target polynucleotide may include one or more (e.g., 1, 2, 3, 4, 5, 6, 7, 8, 9, 10, or more) target sequences, which may be the same or different. In general, different target polynucleotides include different sequences, such as one or more different nucleotides or one or more different target sequences.

The term “template DNA molecule” herein refers to a strand of a nucleic acid from which a complementary nucleic acid strand is synthesized by a DNA polymerase, for example, in a primer extension reaction.

A “portion adjacent to a region of interest” refers to a sequence that is immediately proximal to a region of interest. Reference to a “portion of or adjacent to a region of interest” refers to a sequence that 1) is entirely within the region of interest, 2) is entirely outside but immediately proximal to the region of interest, or 3) includes a contiguous sequence from within and immediately proximal to the region of interest. Reference to a “sequence that is substantially complementary to a portion of or adjacent to a region of interest” refers to 1) a sequence that is substantially complementary to a sequence entirely within the region of interest, 2) a sequence substantially complementary to a sequence entirely outside but immediately proximal to the region of interest, or 3) a sequence that is substantially complementary to a contiguous sequence from with and immediately proximal to the region of interest.

“Noisy Genetic Data” herein refers to genetic data with any of the following: allele dropouts, uncertain base pair measurements, incorrect base pair measurements, missing base pair measurements, uncertain measurements of insertions or deletions, uncertain measurements of chromosome segment copy numbers, spurious signals, missing measurements, other errors, or combinations thereof.

“Confidence” herein refers to the statistical likelihood that the called SNP, SNV, variant, copy number, etc. correctly represents the real genetic state of the individual.

### II. Minimal Residual Disease Detection

The goal of a minimum residual disease (MRD) assay is to detect and/or quantify circulating tumor DNA (ctDNA) so researchers and clinicians can detect recurrence early and monitor the progress of the disease through treatment. In general, an MRD assay will rely on a patient-specific and tumor-specific panel (i.e., a “panel”) for assessing the presence of ctDNA in a patient sample. The panel can be prepared with the general steps of (1) profiling a tumor or cancer sample from a patient, and (2) identifying a subset of somatic mutations to target, and, at one or more later time points, (3) taking a subsequent sample from the patient, (4) enriching cell-free DNA (cfDNA) for the target somatic mutation sites, and (5) determining or estimating the ctDNA content of the cfDNA given the tumor profile and sequencing data.

More specifically, preparing the patient-specific and tumor-specific panel (i.e., a “panel”) may comprise, for example, (a) obtaining a tumor sample and a first non-tumor sample from a cancer patient; (b) sequencing DNA (e.g., genomic DNA) from the tumor sample and sequencing DNA (e.g., genomic DNA) from the first non-tumor sample, thereby obtaining sequences of

DNA or sequence reads from the tumor sample and the non-tumor sample; (c) preparing a preliminary panel comprising a plurality of preliminary tumor-specific somatic mutations that are present in the tumor sample and absent in the first non-tumor sample; (d) obtaining a second non-tumor sample from the patient or reusing the first non-tumor sample; (e) extracting DNA (e.g., genomic DNA) from the second non-tumor sample or first non-tumor sample; (f) enriching the DNA from the second non-tumor sample or first non-tumor sample for sequences corresponding to the preliminary panel; (g) sequencing the enriched DNA fraction to detect the presence or absence of any of the plurality of preliminary tumor-specific somatic mutations; and (h) removing from the preliminary panel any of the plurality of preliminary tumor-specific somatic mutations that are present in the enriched DNA fraction, thereby obtaining a patient-specific panel. Sequencing of the DNA from the tumor sample and non-tumor samples may comprise whole genome sequencing or various types of targeted sequencing, such as whole exome sequencing.

This comparison of the tumor and non-tumor sequences can be performed by, for example, aligning the sequences of DNA from the tumor sample (e.g., genomic DNA) to a reference human genome that is not from the patient and aligning the sequences of DNA from the non-tumor sample (e.g., genomic DNA) to the reference genome that is not from the patient. The reference genome can be, for example, a publicly available human genome assembly, such as hg18, hg19, GRCh38.p14, GRCh37.p13, or other assemblies from the Genome Reference Consortium. Alternatively, the comparison of the tumor and non-tumor sequences can be performed by, for example, aligning the sequences of DNA (e.g., genomic DNA) from the tumor sample to sequences of DNA (e.g., genomic DNA) from the non-tumor sample. With either approach, the skilled artisan is able to detect and identify tumor-specific somatic mutations that are present in the tumor sample but not in the non-tumor sample.

The tumor sample may be a solid tumor sample, such as a biopsy or other tissue sample, or a liquid sample, such as blood (in the case of a hematological cancer) or specific fractions of blood. The non-tumor sample may be tissue-matched with the tumor sample, or it may be from a different tissue. For example, the non-tumor sample may be selected from a healthy (i.e., non-cancerous or non-tumor) tissue sample, blood or specific fractions of blood such as buffy coat, leukocytes, fibroblast, or any other biological sample comprising genomic DNA.

The disclosed methods allow for generating more specific patient-specific panels compared to prior MRD methods by integrating a quality control step to remove other non-tumor somatic mutations from the preliminary panel. As described in further detail below, the method includes extracting DNA from a second non-tumor sample, enriching the DNA from the second-tumor sample for sequencing corresponding to the preliminary panel, sequencing the enriched DNA, and removing from the preliminary panel any of the preliminary tumor-specific somatic mutations that are present in the enriched DNA to obtain a patient-specific panel. Specific aspects of these quality control steps are discussed in more detail below.

Once a patient-specific and tumor-specific panel (i.e., a “panel”) has been established, such a panel can be used to enrich ctDNA (e.g., fragments that include a target sequence corresponding to a tumor-specific somatic mutation or variant) in subsequent samples taken from the cancer patient. The subsequent samples may be taken from a patient at various time points during the course of treatment or during a period of remission. For example, after a surgical removal of a tumor, the tumor may be profiled as described herein to determine tumor-specific somatic mutations, and at one or more subsequent time points a subsequent sample may be taken from the subject to search for the presence of any ctDNA comprising any one of the identified tumor-specific somatic mutations. The detection or presence of ctDNA comprising a tumor-specific somatic mutation may be indicative of cancer recurrence. Additionally or alternatively, similar assessment can be performed throughout the course of a patient's treatment (e.g., with chemotherapy, radiation, immunotherapy, cell therapy, etc.) to detect or quantify ctDNA and determine whether the amount of ctDNA is increasing or decreasing, as this may be indicative of responsiveness to the therapy. Accordingly, assessment of a subsequent sample may be repeated 1, 2, 3, 4, 5, 6, 7, 8, 9, or 10 or more times throughout the course of a patient's remission or treatment. The assessment of a subsequent sample may be repeated monthly, every other month, once every three months, once every four months, once every five months, once every six months, once every seven months, once every eight months, once every nine months, once every ten months, once every eleven months, or annually.

The type of sample used for the one or more subsequent samples is generally a blood sample, a plasma sample, or a serum sample, but any biological sample that contains cfDNA and potentially contains ctDNA would be acceptable. In some embodiments, the one or more subsequent samples are cell-free samples.

Enrichment of ctDNA (e.g., fragments that include a target sequence corresponding to a tumor-specific somatic mutation or variant) in the one or more subsequent samples can be performed by methods including, but not limited to, hybrid capture-based enrichment, PCR-target enrichment, or on-sequencer enrichment. Briefly, enrichment may comprise extracting cfDNA from a subsequent sample taken from the cancer patient and contacting the extracted cfDNA with a plurality of oligonucleotides (i.e., oligonucleotide probes), wherein each oligonucleotide in the plurality of oligonucleotides comprises a nucleic acid sequence that is capable of hybridizing to a cfDNA fragment comprising one of the tumor-specific somatic mutation sequences identified by comparing the sequences of the patients tumor DNA and non-tumor DNA. In some embodiments, the nucleic acid sequence is capable of hybridizing 1 or more nucleotide bases upstream or downstream of the tumor-specific somatic mutation sequences. Thus, enrichment may utilize a set of oligonucleotide probes to selectively enrich ctDNA that may be in the subsequent sample by binding to previously identified tumor-specific somatic mutation sequences.

A panel may comprise 10-5000 tumor-specific somatic mutations. For example, a panel may comprise 10-4000, 10-3000, 10-2500, 10-2000, 10-1500, 10-1000, 10-950, 10-900, 10-850, 10-800, 10-750, 10-700, 10-650, 10-600, 10-550, 10-500, 50-5000, 50-4000, 50-3000, 50-2500, 50-2000, 50-1500, 50-1000, 50-950, 50-900, 50-850, 50-800, 50-750, 50-700, 50-650, 50-600, 50-550, 50-500, 100-5000, 100-4000, 100-3000, 100-2500, 100-2000, 100-1500, 100-1000, 100-950, 100-900, 100-850, 100-800, 100-750, 100-700, 100-650, 100-600, 100-550, 100-500, 200-5000, 200-4000, 200-3000, 200-2500, 200-2000, 200-1500, 200-1000, 200-950, 200-900, 200-850, 200-800, 200-750, 200-700, 200-650, 200-600, 200-550, 200-500, 300-5000, 300-4000, 300-3000, 300-2500, 300-2000, 300-1500, 300-1000, 300-950, 300-900, 300-850, 300-800, 300-750, 300-700, 300-650, 300-600, 300-550, 300-500, 400-5000, 400-4000, 400-3000, 400-2500, 400-2000, 400-1500, 400-1000, 400-950, 400-900, 400-850, 400-800, 400-750, 400-700, 400-650, 400-600, 400-550, 400-500, 500-5000, 500-4000, 500-3000, 500-2500, 500-2000, 500-1500, 500-1000, 500-950, 500-900, 500-850, 500-800, 500-750, 500-700, 500-650, 500-600, or 500-550 tumor-specific somatic mutations. In some embodiments, a panel may comprise or consist of about 10, about 20, about 30, about 40, about 50, about 75, about 100, about 150, about 200, about 250, about 300, about 350, about 400, about 450, about 500, about 550, about 600, about 650, about 700, about 750, about 800, about 850, about 900, about 950, about 1000, about 1100, about 1150, about 1200, about 1250, about 1300, about 1350, about 1400, about 1450, about 1500, about 1550, about 1600, about 1650, about 1700, about 1750, about 1800,about 1850, about 1900, about 1950, or about 2000 or more tumor-specific somatic mutations. In some embodiments, a panel may comprise at least 10, at least 20, at least 30, at least 40, at least 50, at least 75, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1100, at least 1150, at least 1200, at least 1250, at least 1300, at least 1350, at least 1400, at least 1450, at least 1500, at least 1550, at least 1600, at least 1650, at least 1700, at least 1750, at least 1800, at least 1850, at least 1900, at least 1950, or at least 2000 tumor-specific somatic mutations. The tumor-specific somatic mutations may be in introns, exons, or a combination thereof.

After enrichment or concurrently with enrichment of ctDNA (e.g., fragments that include target sequence corresponding to a tumor-specific somatic mutation or variant), the enriched DNA is sequenced. This sequencing may be performed by, for example Next Generation Sequencing (NGS). Deep sequencing may allow for more sensitive detection, and so the depth of the sequencing may be at least 50×, at least 100×, at least 150×, at least 200×, at least 250×, at least 300×, at least 350×, at least 400×, at least 450×, at least 500×, at least 550×, at least 600×, at least 650×, at least 700×, at least 750×, at least 800×, at least 850×, at least 900×, at least 950×, or at least 1000X. In other words, the depth of the sequencing may be about 50×,about 100×, about 150×, about 200×, about 250×, about 300×, about 350×, about 400×, about 450×, about 500×, about 550×, about 600×, about 650×, about 700×, about 750×, about 800×,about 850×, about 900×, about 950×, or about 1000X. The detection sensitivity of the disclosed methods may be about 20 to about 50 ctDNA fragments comprising one or more of the set of somatic mutations in the fluid sample per a total background of about 500,000 cfDNA fragments.

The disclosed methods may be used for tracking and assessing recurrence in any cancer patient. For example, the cancer patient may have a cancer selected from, but not limited to, adrenal cancer, anal cancer, bile duct cancer, bladder cancer, bone cancer, a brain/CNS tumor, breast cancer, Castleman disease, cervical cancer, colon or rectum cancer, endometrial cancer, esophagus cancer, a Ewing tumor, eye cancer, gallbladder cancer, a gastrointestinal carcinoid tumor, a gastrointestinal stromal tumor (GIST), gestational trophoblastic disease, Hodgkin disease, Kaposi sarcoma, kidney cancer, laryngeal and hypopharyngeal cancer, leukemia, liver cancer, lung cancer, lymphoma, malignant mesothelioma, multiple myeloma, myelodysplastic Syndrome, nasal cavity or paranasal sinus cancer, nasopharyngeal cancer, neuroblastoma, oral cavity or oropharyngeal cancer, osteosarcoma, ovarian cancer, pancreatic cancer, penile cancer, a pituitary tumor, prostate cancer, retinoblastoma, rhabdomyosarcoma, salivary gland cancer, skin cancer, small intestine cancer, stomach cancer, testicular cancer, thymus cancer, thyroid cancer, uterine sarcoma, vaginal cancer, vulvar cancer, Waldenstrom macroglobulinemia, and Wilms tumor. In some embodiments, the cancer may be a blood borne or hematological cancer such as leukemia or lymphoma.

The disclosed MRD assay, specifically the obtaining and testing of subsequent samples from a cancer patient, may be repeated one or more times following completion of a cancer treatment; one or more times while the cancer patient is in remission; one or more times coinciding with or prior to surgery; following, during, or prior to administration of chemotherapy; following, during, or prior to radiation therapy; following, during, or prior to immunotherapy; or following, during, or prior to cell therapy. The disclosed MRD assay may also be repeated at times prior to, coinciding with, and/or following an imaging test, such as a PET scan, a PET/CT scan, an MRI, or an X-ray.

The disclosed methods allow for detecting ctDNA or determining the tumor fraction from a biological sample from a patient that has, previously had, or is suspected of having cancer. As described in further detail below, the methods can be represented by two phases. In a first phase, or enrollment phase, somatic mutations that are specific to a patient are identified, and then filtered to generate a subset of somatic mutations that include only specific types of somatic mutations or show a preference for specific types of somatic mutations. For the purposes of the disclosed methods, the subset of somatic mutations may comprise or consist of multi-nucleotide variants, small indels, and genomic rearrangements for the reasons described herein. A panel of capture probes is then generated that are specific to the subset panel of somatic mutations, which can be used to enrich a sample before sequencing.

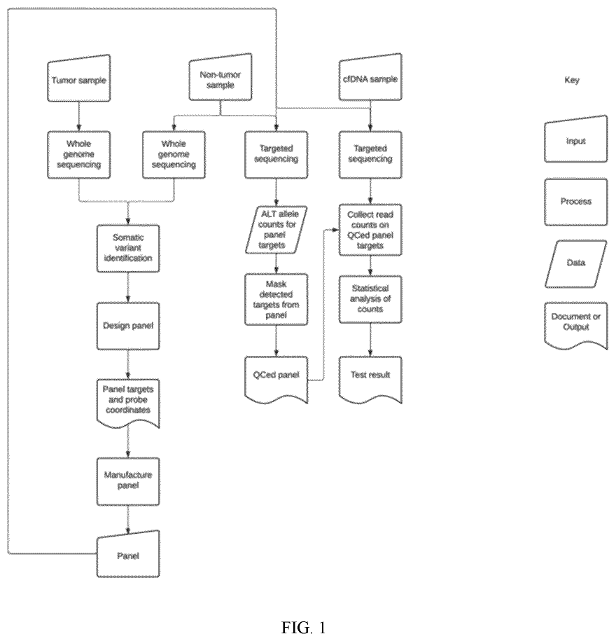

An exemplary workflow of the disclosed methods is provided in FIG. 1.

Specific aspects of MRD processes are discussed in more detail below.

### III. Phase I—Panel of Markers/Mutations and Capture Probes

a. DNA Library Preparation

In some embodiments of the methods disclosed herein, a DNA library is obtained or prepared from DNA obtained from a patient, e.g., a cancer patient. In some embodiments, a DNA library is obtained or prepared from the genome of the patient. In some embodiments, the DNA has been previously sequenced and mutations or variants identified.

When producing a DNA library from genomic DNA, the genomic DNA can be fragmented, for example by using a hydrodynamic shear or other mechanical force, or fragmented by chemical or enzymatic digestion, such as restriction digesting. This fragmentation process allows the DNA molecules present in the genome to be sufficiently short for analysis, such as sequencing or digital PCR. cfDNA, however, is generally sufficiently short such that no fragmentation is necessary. cfDNA originates from genomic DNA. A portion of the cfDNA obtained from a plasma sample of a cancer patient may originate from cancer cells (i.e., circulating tumor DNA or ctDNA), and a portion of the cfDNA may originate from non-cancer cells.

In some embodiments, the DNA molecules are subjected to additional modification, resulting in the attachment of oligonucleotides to the DNA molecules. The oligonucleotides can comprise an adapter sequence or a molecular barcode (or both). In some embodiments, the adapter sequence is common to all oligonucleotides in a plurality of oligonucleotides that are used to form the DNA library. In some embodiments, the molecular barcodes are unique or have low redundancy. By way of example, the oligonucleotide can be attached to the DNA molecules by ligation. Direct attachment of the oligonucleotides to the DNA molecules in the DNA library can be used, for example, when enrichment occurs in a downstream process. For example, in some embodiments, a DNA library is prepared by direct attachment of an oligonucleotide comprising a molecular barcode and an adapter sequence, followed by enrichment (for example, by hybridization) of DNA molecules comprising a region of interest or a portion of a region of interest.

In some embodiments, library preparation and enrichment occur simultaneously. For example, in some embodiments, DNA molecules comprising a region of interest or a portion thereof are preferentially amplified. This can be done, for example, by combining the cfDNA (or genomic DNA), with oligonucleotides comprising a target-specific sequence, an adapter sequence, and a molecular barcode, and amplifying the DNA molecules. As before, in some embodiments, the adapter sequence is common to all oligonucleotides in a plurality of oligonucleotides, and the molecular barcode is unique or of low redundancy. The target-specific sequence is unique to the targeted region of interest or portion thereof. Thus, PCR amplification selectively amplifies the DNA molecules comprising the region of interest or portion thereof.

When the methods include the use of tags or molecular barcodes, the tag or molecular barcode may also be ligated to the fragments or included within the ligated adapter sequences. The independent attachment of the tag or molecular barcode, as opposed to incorporating the tag or molecular barcode, may vary with the enrichment method. For example, when using hybrid capture-based target enrichment the adapter can include the molecular barcode, when using PCR-targeted enrichment target-specific primer pairs and overhangs are used that will incorporate the sequencing adapters and sample-specific and molecular barcodes, and when using on-sequencer enrichment the adapter may be separately ligated from the tag or molecular barcode.

b. Panel of Mutations/Markers

In some embodiments, sequencing of the nucleic acid from the sample is performed using whole genome sequencing (WGS). In some embodiments, targeted sequencing is performed and may be either DNA or RNA sequencing. The targeted sequencing may be to a subset of the whole genome. In some embodiments the targeted sequencing is to introns, exons, non-coding sequences or a combination thereof. In other embodiments, targeted whole exome sequencing (WES) of the DNA from the sample is performed. The DNA is sequenced using a next generation sequencing platform (NGS), which is massively parallel sequencing. NGS technologies provide high throughput sequence information, and provide digital quantitative information, in that each sequence read that aligns to the sequence of interest is countable. In certain embodiments, clonally amplified DNA templates or single DNA molecules are sequenced in a massively parallel fashion within a flow cell. In addition to high-throughput sequence information, NGS provides quantitative information, in that each sequence read is countable and represents an individual clonal DNA template or a single DNA molecule. The sequencing technologies of NGS include pyrosequencing, sequencing-by-synthesis with reversible dye terminators, sequencing by oligonucleotide probe ligation and ion semiconductor sequencing. DNA from individual samples can be sequenced individually (i.e., singleplex sequencing) or DNA from multiple samples can be pooled and sequenced as indexed genomic molecules (i.e., multiplex sequencing) on a single sequencing run, to generate up to several hundred million reads of DNA sequences. Commercially available platforms include, e.g., platforms for sequencing-by-synthesis, ion semiconductor sequencing, pyrosequencing, reversible dye terminator sequencing, sequencing by ligation, single-molecule sequencing, sequencing by hybridization, and nanopore sequencing. Platforms for sequencing by synthesis are available from, e.g., Illumina, 454 Life Sciences, Helicos Biosciences, and Qiagen. Illumina platforms can include, e.g., Illumina's Solexa platform, Illumina's Genome Analyzer. Life Science platforms include, e.g., the GS Flex and GS Junior, and are described in U.S. Pat. No. 7,323,305. Platforms from Helicos Biosciences include the True Single Molecule Sequencing platform. Ion Torrent, an alternative NGS system, is available from ThermoScientific and is a semiconductor based technology that detects hydrogen ions that are released during polymerization of nucleic acids. Any detection method that allows for the detection of segregating markers may be used with the assay provided for herein.

In some embodiments, whole genome sequencing (WGS) of the tumor and normal DNA is performed.

In other embodiments, Whole Exome Sequencing (WES) of the tumor and normal DNA is performed. WES comprises selecting DNA sequences that encode proteins, and sequencing that DNA using any high throughput DNA sequencing technology. Methods that can be used to target exome DNA include the use of polymerase chain reaction (PCR), molecular inversion probes (MIP), hybrid capture, and in-solution capture. The utility of targeted genome approaches is well established, and commercially available methods for WES include the Roche NimbleGen Capture Array (Roche NimbleGen Inc., Madison, WI), Agilent SureSelect (Agilent Technologies, Santa Clara, CA), and RainDance Technologies emulsion PCR (RainDance Technologies, Lexington, MA), IDT xGen® Exome Research Panel and others.

Sequence reads may comprise about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, about 500 bp, or more than 500 bp.

In some embodiments of the methods described herein, the somatic mutations identified will be analyzed and filtered to generate a subset panel of markers. For example, the subset panel of markers may comprise one or more types of somatic mutation, including but not limited to single-nucleotide variants (SNVs) multi-nucleotide variants, insertions and deletions (e.g., indel variants), and genomic rearrangements. In some embodiments, the subset panel will only include somatic mutations that comprise multiple changes compared to the normal sample, i.e., the subset panel will not include any SNVs. In some embodiments, the subset panel of somatic mutations can include greater than 50, up to 100, up to 200, up to 300, up to 400, up to 500, up to 600, up to 700, up to 800, up to 900, up to 1,000, up to 1,500, up to 2,000, up to 2,500, up to 3,000, up to 4,000, up to 5,000, up to 6,000, up to 7,000, up to 8,000, up to 9,000, up to 10,000, up to 11, 000, up to 12,000, up to 13,000, up to 14,000, up to 15,000, or more than 15,000 mutations, which may comprise MNVs, small indels, genomic rearrangements, or combinations thereof. In other embodiments, the subset panel includes between 50 and 15,000 mutations, between 100 and 15,000 mutations, between 500 and 13,000 mutations, between 1,000 and 10,000 mutations, between 2,000 and 8,000 mutations, or between 4,000 and 6,000 mutations.

c. Capture Probes

The subset panel is represented by a set of oligonucleotide capture probes each designed to at least partially hybridize to a target sequence that has been identified to comprise a mutation identified in the tumor sample from the patient or in the parental sequence. In some embodiments, the subset panel comprises capture probes comprising the subset of somatic mutations identified in the patient's tumor. In some embodiments, each capture probe is designed to selectively hybridize to a target sequence. The capture probe can be at least 70%, 75%, 80%, 90%, 95%, or more than 95% complementary to a target sequence. In some embodiments, the capture probe is 100% complementary to a target sequence. In some embodiments the capture probes are DNA probes. In other embodiments, the capture probes can be RNA.

The capture probe generally is sufficiently long to encompass the sequence of a somatic mutation, or corresponding normal sequence comprised in the genomic sequence targeted by the capture probe. The length and composition of a capture probe can depend on many factors including temperature of the annealing reaction, source and base composition of the oligonucleotide, and the estimated ratio of probe to genomic target sequence. Additionally, the length of the capture probe is dependent on the length of the target sequence it is designed to capture. The method provided utilizes cfDNA including circulating tumor DNA (ctDNA) as the source of the target sequences that are to be captured. Accordingly, as cfDNA is highly fragmented to an average of about 170 bp, the capture probe can be, for example, between 100 and 300 bp, between 150 and 250 bp, or between 175 and 200 bp. Currently, methods known in the art describe probes that are typically longer than 120 bases. In a current embodiment, if the allele is one or a few bases then the capture probes may be less than about 110 bases, less than about 100 bases, less than about 90 bases, less than about 80 bases, less than about 70 bases, less than about 60 bases, less than about 50 bases, less than about 40 bases, less than about 30 bases, and less than about 25 bases, and this is sufficient to ensure equal enrichment from all alleles. When the mixture of DNA that is to be enriched using the hybrid capture technology is a mixture comprising cfDNA isolated from blood the average length of DNA is quite short, typically less than 200 bases. The use of shorter probes results in a greater chance that the hybrid capture probes will capture desired DNA fragments. Larger variations may require longer probes. For the purposes of the present disclosure, the variations of interest are more than one base in length. In some embodiments, targeted regions in the genome can be preferentially enriched using hybrid capture probes wherein the hybrid capture probes are shorter than 90 bases, and can be less than 80 bases, less than 70 bases, less than 60 bases, less than 50 bases, less than 40 bases, less than 30 bases, or less than 25 bases. In some embodiments, to increase the chance that the desired allele is sequenced, the length of the probe that is designed to hybridize to the regions flanking the polymorphic allele location can be decreased from above 90 bases, to about 80 bases, or to about 70 bases, or to about 60 bases, or to about 50 bases, or to about 40 bases, or to about 30 bases, or to about 25 bases.

Hybrid capture probes can be designed such that the region of the capture probe with DNA that is complementary to the DNA found in regions flanking the polymorphic allele is not immediately adjacent to the polymorphic site. Instead, the capture probe can be designed such that the region of the capture probe that is designed to hybridize to the DNA flanking the polymorphic site of the target is separated from the portion of the capture probe that will be in van der Waals contact with the polymorphic site by a small distance that is equivalent in length to one or a small number of bases. In an embodiment, the hybrid capture probe is designed to hybridize to a region that is flanking the polymorphic allele but does not cross it; this may be termed a flanking capture probe. The length of the flanking capture probe may be less than about 120 bases, less than about 110 bases, less than about 100 bases, less than about 90 bases, and can be less than about 80 bases, less than about 70 bases, less than about 60 bases, less than about 50 bases, less than about 40 bases, less than about 30 bases, or less than about 25 bases. The region of the genome that is targeted by the flanking capture probe may be separated by the polymorphic locus by 1, 2, 3, 4, 5, 6, 7, 8, 9, 10, 11-20, or more than 20 base pairs.

For small insertions or deletions, one or more probes that overlap the mutation may be sufficient to capture and sequence fragments comprising the mutation. Hybridization may be less efficient between the probe-limiting capture efficiency, typically designed to the reference genome sequence. To ensure capture of fragments comprising the mutation one could design two probes, one matching the normal allele and one matching the mutant allele. A longer probe may enhance hybridization. Multiple overlapping probes may enhance capture. Finally, placing a probe immediately adjacent to, but not overlapping, the mutation may permit relatively similar capture efficiency of the normal and mutant alleles.

For Short Tandem Repeats (STRs), a probe overlapping these highly variable sites is unlikely to capture the fragment well. To enhance capture a probe could be placed adjacent to, but not overlapping the variable site. The fragment could then be sequenced as normal to reveal the length and composition of the STR.

For large deletions, a series of overlapping probes, a common approach currently used in exon capture systems may work. However, with this approach it may be difficult to determine whether or not an individual is heterozygous. According to the method provided, custom probes are designed to ensure capture of the unique set of somatic mutations identified in the patient's tumor.

Capture probes can be modified to comprise purification moieties that serve to isolate the capture duplex from the unhybridized, untargeted cfDNA sequences by binding to a purification moiety binding partner. Suitable binding pairs for use in the invention include, but are not limited to, antigens/antibodies (for example, digoxigenin/antidigoxigenin, dinitrophenyl (DNP)/anti-DNP, dansyl-X-antidansyl, Fluorescein/anti-fluorescein, lucifer yellow/anti-lucifer yellow, and rhodamine anti-rhodamine); biotin/avidin (or biotin/streptavidin); calmodulin binding protein (CBP)/calmodulin; hormone/hormone receptor; lectin/carbohydrate; peptide/cell membrane receptor; protein A/antibody; hapten/antihapten; enzyme/cofactor; and enzyme/substrate. Other suitable binding pairs include polypeptides such as the FLAG-peptide (Hopp et al., BioTechnology, 6:1204-1210 (1988)); the KT3 epitope peptide (Martin et al., Science, 255:192-194 (1992)); tubulin epitope peptide (Skinner et al., J. Biol. Chem., 266:15163-15166 (1991)); and the T7 gene 10 protein peptide tag (Lutz-Freyermuth et al., Proc. Natl. Acad. Sci. USA, 87:6393-6397 (1990)) and the antibodies each thereto. Further non-limiting examples of binding partners include agonists and antagonists for cell membrane receptors, toxins and venoms, viral epitopes, hormones such as steroids, hormone receptors, peptides, enzymes and other catalytic polypeptides, enzyme substrates, cofactors, drugs including small organic molecule drugs, opiates, opiate receptors, lectins, sugars, saccharides including polysaccharides, proteins, and antibodies including monoclonal antibodies and synthetic antibody fragments, cells, cell membranes and moieties therein including cell membrane receptors, and organelles. In some embodiments, the first binding partner is a reactive moiety, and the second binding partner is a reactive surface that reacts with the reactive moiety, such as described herein with respect to other aspects of the invention. In some embodiments, the oligonucleotide primers are attached to the solid surface prior to initiating the extension reaction. Methods for the addition of binding partners to capture oligonucleotide probes are known in the art, and include addition during (such as by using a modified nucleotide comprising the binding partner) or after synthesis. Additionally, the capture probes can be tethered to a solid surface, e.g., a magnetic bead, which facilitates the isolation of captured sequences.

### IV. Phase II-Detection and Monitoring Tumors by Analyzing cfDNA

a. Targeted Enrichment of a Region of Interest

The disclosed methods generally comprise enriching a target sequence in a region of interest. Examples of enrichment techniques include, but are not limited to, hybrid capture, selective circularization (also referred to as molecular inversion probes (MIP)), and PCR amplification of targeted regions of interest. Hybrid capture methods are based on the selective hybridization of the target genomic regions to user-designed oligonucleotides. The hybridization can be to oligonucleotides immobilized on high or low density microarrays (on-array capture), or solution-phase hybridization to oligonucleotides modified with a ligand (e.g., biotin) which can subsequently be immobilized to a solid surface, such as a bead (in-solution capture). Molecular inversion probe (MIP)-based method relies on construction of numerous single-stranded linear oligonucleotide probes, consisting of a common linker flanked by target-specific sequences. Upon annealing to a target sequence, the probe gap region is filled via polymerization and ligation, resulting in a circularized probe. The circularized probes are then released and amplified using primers directed at the common linker region. PCR-based methods employ highly parallel PCR amplification, where each target sequence in the sample has a corresponding pair of unique, sequence-specific primers. In some embodiments, enrichment of a target sequence occurs at the time of sequencing.

In the second phase of the method, samples that are used for determining the tumor fraction of the patient include samples that contain nucleic acids that are cell-free. Cell-free nucleic acids, including cfDNA, can be obtained by various methods from biological samples including but not limited to plasma, serum, and urine. Other biological fluid samples include, but are not limited to blood, sweat, tears, sputum, car flow, lymph, saliva, cerebrospinal fluid, ravages, bone marrow suspension, vaginal flow, transcervical lavage, brain fluid, ascites, milk, secretions of the respiratory, intestinal and genitourinary tracts, amniotic fluid, milk, and leukapheresis samples. In some embodiments, the sample is a sample that is easily obtainable by non-invasive procedures, e.g., blood, plasma, serum, sweat, tears, sputum, urine, car flow, saliva, or feces. In certain embodiments the sample is a peripheral blood sample, or the plasma and/or serum fractions of a peripheral blood sample. In other embodiments, the biological sample is a swab or smear, a biopsy specimen, or a cell culture. In another embodiment, the sample is a mixture of two or more biological samples, e.g., a biological sample can comprise two or more of a biological fluid sample, a tissue sample, and a cell culture sample.

In various embodiments, the cfDNA present in the sample can be enriched specifically or non-specifically prior to use (e.g., prior to capture and sequencing). Non-specific enrichment of sample DNA refers to the whole genome amplification of the DNA fragments of the sample that can be used to increase the level of the sample DNA prior to capture and sequencing. Non-specific enrichment can be the selective enrichment of exomes. Methods for whole genome amplification are known in the art. Degenerate oligonucleotide-primed PCR (DOP), primer extension PCR technique (PEP) and multiple displacement amplification (MDA) are examples of whole genome amplification methods. In some embodiments, the sample is unenriched for cfDNA.

As is described elsewhere herein, cfDNA is present as fragments averaging about 170 bp. Accordingly, further fragmentation of cfDNA is not needed. In some embodiments, sufficient cfDNA is obtained from a 10 ml blood sample to confidently determine the presence or absence of cancer in a patient. The blood samples used in the method provided can be of about 5 ml, about 10 ml, about 15 ml, about 20 ml, about 25 ml or more than 25 ml. Typically, 20 ml of blood plasma contains between 5,000 and 10,000 genome equivalents, and provides more than sufficient cfDNA for determining tumor fraction according to the method provided. In some embodiments, sufficient cfDNA is obtained from 10 ml to 20 ml of blood to determine tumor fraction.

To separate cfDNA from cells in a sample, various methods including, but not limited to fractionation, centrifugation (e.g., density gradient centrifugation), DNA-specific precipitation, or high-throughput cell sorting and/or other separation methods can be used. Commercially available kits for manual and automated separation of cfDNA are available (Roche Diagnostics, Indianapolis, Ind., Qiagen, Germantown, MD).

cfDNA can be end-repaired, and optionally dA tailed, and double-stranded adaptors comprising sequences complementary to amplification and sequencing primers are ligated to the ends of the cfDNA molecules to enable NGS sequencing, e.g., using an Illumina platform. Additionally, each of the double-stranded adaptors further comprises a non-random barcode sequence, which serves to differentiate individual cfDNA molecules. In some embodiments, the barcode sequences are random sequences. In other embodiments, the barcode sequences are non-random barcode sequences. Non-random barcode sequences provide a significant advantage over random barcode sequences because non-random barcode sequences enable unambiguous identification of the sequencing reads described below. The nonrandom barcode sequences are designed specifically to be base-balance both within and across all barcodes. Additionally, in some embodiments, the nonrandom barcodes can comprise a T nucleotide at the 3′ end, which is complementary to the A nucleotide of dA-tailed cfDNA molecules. In embodiments utilizing a T nucleotide overhang at the 3′ end of the barcode, barcodes of three different lengths can be designed to avoid a single base flashing across the entire flowcell of the sequencer. Nonrandom barcode sequences can be present in adaptors as sequences of 13, 14, and 15 bp; 10, 11, and 12 bp; 11, 12, and 13 bp; 13, 14, and 15 bp; 14, 15, and 16 bp; 15, 16, and 17 bp, and the like. In some embodiments, the shortest barcode sequence can be 8 bp and the longest barcode sequence can be 100 bp.

Each sequence of the subpanel that is present in the cfDNA sample is targeted by one or more capture probes described elsewhere herein, and is isolated for further analysis.

b. Sequencing and Analysis

The disclosed methods generally comprise sequencing one or more samples. Sequencing methods include, but are not limited to, Maxam-Gilbert sequencing-based techniques, chain-termination-based techniques, shotgun sequencing, bridge PCR sequencing, single-molecule real-time sequencing, ion semiconductor sequencing (Ion Torrent sequencing), nanopore sequencing, pyrosequencing (454), sequencing by synthesis, sequencing by ligation (SOLiD sequencing), sequencing by electron microscopy, dideoxy sequencing reactions (Sanger method), massively parallel sequencing, polony sequencing, duplex sequencing, and DNA nanoball sequencing. In some embodiments, sequencing involves hybridizing a primer to the template to form a template/primer duplex, contacting the duplex with a polymerase enzyme in the presence of a detectably labeled nucleotides under conditions that permit the polymerase to add nucleotides to the primer in a template-dependent manner, detecting a signal from the incorporated labeled nucleotide, and sequentially repeating the contacting and detecting steps at least once, wherein sequential detection of incorporated labeled nucleotide determines the sequence of the nucleic acid. In some embodiments, the sequencing comprises obtaining paired end reads. The accuracy or average accuracy of the sequence information may be greater than 80%, 90%, 95%, 99% or 99.98%. In some embodiments, the sequence information obtained is more than 50 bp, 100 bp or 200 bp. The sequence information may be obtained in less than 1 month, 2 weeks, 1 week 1 day, 3 hours, 1 hour, 30 minutes, 10 minutes, or 5 minutes. The sequence accuracy or average accuracy may be greater than 95% or 99%. Examples of detectable labels include radiolabels, florescent labels, enzymatic labels, etc. In some embodiments, the detectable label may be an optically detectable label, such as a fluorescent label. Examples of fluorescent labels include cyanine, rhodamine, fluorescein, coumarin, BODIPY, alexa, or conjugated multi-dyes. In some embodiments, the nucleotide is flagged if one or more of its sequence segments are substantially similar to one or more sequence segments of another nucleotide within the same partition.

Some methods of sequencing may require or involve a prior target enrichment step. For example, use of on-sequencer enrichment, such as with a nanopore sequencer, allows for the simultaneous enrichment and sequencing of the sequence library by real-time rejection of molecules that are not from the region of interest. Alternatively, sequences can be selectively and preferentially sequenced from the region of interest.

Captured sequences can be analyzed using the sequencing-by-synthesis technology of Illumina, which uses fluorescent reversible terminator deoxyribonucleotides. The reads generated by the sequencing process are aligned to a reference sequence and associated with a sequence of the somatic sequence panel specific for the patient. Mapping of the sequence reads can be achieved by comparing the sequence of the reads with the sequence of the reference genome to determine the specific genetic information, and optionally the chromosomal origin of the sequenced nucleic acid (e.g., cfDNA) molecule. A number of computer algorithms are available for aligning sequences, including without limitation BLAST (Altschul et al., 1990), BLITZ (MPsrch) (Sturrock & Collins, 1993), FASTA (Person & Lipman, 1988), BOWTIE (Langmead et al, Genome Biology 10: R25.1-R25.10 [2009]), or ELAND (Illumina, Inc., San Diego, Calif., USA). In one embodiment, the sequencing data is processed by bioinformatic alignment analysis for the Illumina Genome Analyzer, which uses the Efficient Large-Scale Alignment of Nucleotide Databases (ELAND) software. Additional software includes SAMtools (SAMtools, Bioinformatics, 2009, 25(16):2078-9), and the Burroughs-Wheeler block sorting compression procedure which involves block sorting or preprocessing to make compression more efficient.

The barcoded cfDNA fragments isolated from the patient's fluid sample, e.g., blood sample, can be amplified, e.g., by PCR, and captured using the hybrid probes. Capturing of the barcoded fragments comprises obtaining single strands of barcoded cfDNA, and hybridizing the barcoded cfDNA with different hybrid probes. Each of the different hybrid probes hybridizes to a single-stranded barcoded cfDNA target sequence to form a target-hybrid probe duplex. The duplex is isolated from unhybridized cfDNA by binding the purification binding moiety comprised in the hybrid probe to the corresponding purification moiety binding partner. As described elsewhere herein, the corresponding purification moiety binding partner can be immobilized on a solid surface, e.g., a magnetic bead, which facilitates the separation of the capture duplex from unhybridized cfDNA molecules in solution. The barcoded cfDNA of the duplex is released, and is subjected to sequencing using an NGS instrument.

The error rate in sequencing using NGS methods is of approximately 1 in 500 bases which results in many sequencing errors. The high error rate becomes problematic especially when attempting to identify somatic mutations in mixtures of DNA sequences comprising only a small fraction of mutated species or sequences comprising single nucleotide variants. The methods described herein avoid such errors by analyzing target sequences that comprise somatic mutations having multiple changes relative to a reference sequence. Additionally, NGS methods typically utilize single stranded DNA as the primary source of sequencing material. Any error included during the amplification step of the DNA molecule prior to sequencing is perpetuated, and becomes indistinguishable as an extraneous technology-dependent mistake. Chemical errors occur at a frequency of approximately in 1000 bases. The combination of sequencing and chemical errors obscures the limit of detection (LOD).

Accordingly, in some embodiments, double-stranded sequencing of the cfDNA is performed. As described elsewhere herein, cfDNA can be end-repaired, and optionally dA tailed, and double-stranded adaptors comprising sequences complementary to amplification and sequencing primers are ligated to the ends of the cfDNA molecules to enable NGS sequencing, e.g., using an Illumina platform.

The tumor fraction can then be calculated as the proportion of different cfDNA sequences each comprising at least one somatic mutation, i.e., ctDNA sequences, relative to the total number of different cfDNA, i.e., ctDNA and corresponding normal sequences. Unlike the single-stranded approach, the current method corrects for random sequencing errors.

c. Molecular Barcodes

In some embodiments, an identifier sequence, i.e., a molecular barcode, may be used to identify unique DNA molecules or target sequences in a DNA library. Molecular barcodes aid in reconstruction of a contiguous DNA sequences or assist in copy number variation determination. Exemplary markers include nucleic acid binding proteins, optical labels, nucleotide analogs, nucleic acid sequences, and others known in the art.

In some embodiments, the molecular barcode is a nanostructure barcode. In some embodiments, the molecular barcode comprises a nucleic acid sequence that when joined to a target polynucleotide serves as an identifier of the sample or sequence from which the target polynucleotide was derived. In some embodiments, molecular barcodes are at least 3, 4, 5, 6, 7, 8, 9, 10, 11, 12, 13, 14, 15, or more nucleotides in length. In some embodiments, molecular barcodes are shorter than 10, 9, 8, 7, 6, 5, or 4 nucleotides in length. In some embodiments, each molecular barcode in a plurality of molecular barcodes differ from every other molecular barcode in the plurality at least three nucleotide positions, such as at least 3, 4, 5, 6, 7, 8, 9, 10, or more positions. In some embodiments, molecular barcodes associated with some polynucleotides are of different length than molecular barcodes associated with other polynucleotides. In general, molecular barcodes are of sufficient length and comprise sequences that are sufficiently different to allow the identification of samples based on molecular barcodes with which they are associated. In some embodiments, both the forward and reverse adapter comprise at least one of a plurality of molecular barcode sequences. In some embodiments, each reverse adapter comprises at least one of a plurality of molecular barcode sequences, wherein each molecular barcode sequence of the plurality of molecular barcode sequences differs from every other molecular barcode sequence in the plurality of molecular barcode sequences.

In some embodiments, every molecular barcode in a set is unique, that is, any two molecular barcodes chosen out of a given set will differ in at least one nucleotide position. Furthermore, it is contemplated that molecular barcodes have certain biochemical properties that are selected based on how the set will be used. For example, certain sets of molecular barcodes that are used in an RT-PCR reaction should not have complementary sequences to any sequence in the genome of a certain organism or set of organisms. A requirement for non-complementarity helps to ensure that the use of a particular molecular barcode sequence will not result in mis-priming during molecular biological manipulations requiring primers, such as reverse transcription or PCR. Certain sets satisfy other biochemical properties imposed by the requirements associated with the processing of the sequence molecules into which the barcodes are incorporated.

Examples of sequencing technologies for sequencing molecular barcodes, as well as any generated nucleotide-based sequence, include, but are not limited to, Maxam-Gilbert sequencing-based techniques, chain-termination-based techniques, shotgun sequencing, bridge PCR sequencing, single-molecule real-time sequencing, ion semiconductor sequencing (Ion Torrent sequencing), nanopore sequencing, pyrosequencing (454), sequencing by synthesis, sequencing by ligation (SOUD sequencing), sequencing by electron microscopy, dideoxy sequencing reactions (Sanger method), massively parallel sequencing, polony sequencing, and DNA nanoball sequencing.

In some embodiments, molecular barcodes are used to improve the power of copy-number calling algorithms by reducing non-independence from PCR duplication. In another embodiment, molecular barcodes can be used to improve test specificity by reducing sequence error generated during amplification.

### V. Method of Generating Patient-Specific Panels

Detecting somatic mutations that are present in a low frequency through next generation sequencing (NGS) presents a promising method of detecting disease presence, including cancer reoccurrence, early and through less invasive procedures. Deep sequencing is an NGS approach that is highly sensitive and can detect rare mutations or polymorphisms from a sample. Deep sequencing relies upon a high depth of coverage, meaning the process will generate a high number of reads of a given nucleotide sequence in a single experiment. By increasing the depth of coverage by increasing the number of reads of the given sequence, it will be possible to detect rare mutations or polymorphisms in a sample containing many copies of the same nucleotide sequence by increasing the likelihood that the mutated sequence will be amplified and detected. Thus, tools utilizing NGS are much more sensitive and able to detect even very rare mutations in a given sample.

While the power of deep sequencing is in its depth of coverage, a major issue is similarly based upon this feature. Because there is such a high depth of coverage, any errors in initial sequence identification can be amplified and could produce a false positive result. For example, two cells from the same individual may have different background mutations that are not associated with any disease phenotype as non-harmful mutations are known to accumulate in different cell types or cell lineages that divide frequently. Comparing sequences from a known tumor sample and a non-tumor sample a single time may not truly identify mutations associated with the disease and those that are simply rare, non-harmful background mutations, This is because the single non-tumor sample may not contain all non-harmful background mutations present in the individual. As NGS detects even rare sequences present in a sample, it is likely that sequencing a future sample from the individual would uncover rare non-harmful mutations in different cells of the individual's body. This would result in a false positive result if the non-harmful mutation was improperly associated with the disease phenotype, meaning that the individual would believe that their disease has either returned or has not responded to treatment. This could lead to the individual undergoing costly, invasive, and potentially dangerous diagnostic procedures or treatments based on the incorrect result. To ensure that the method is most effective, it is integral to reduce the likelihood of generating false positives by ensuring only disease-related mutations are used in a patient's panel.

One method of eliminating false positives is to include in the patient-specific panel only variants that include multiple changes relative to a reference sequence. It would be unlikely that the variant would be observed only due to an assay error, sequencing error, or single background mutations in the genome. While effective, this method may eliminate certain true disease-related mutations from inclusion in the panel, preventing the effective detection of tumor cells. Therefore, additional methods to enhance the sensitivity and accuracy of such methods is necessary.

The present technology addresses this issue by integrating additional quality control to reduce the likelihood of incorporating mutations not associated with the presence of a disease in the MRD panel. Disclosed herein is a method for detection and/or quantitation of ctDNA that includes a step of screening a preliminary panel against a second non-tumor sample. For the purposes of the present disclosure, the first non-tumor sample and the second non-tumor sample may be the same sample. For example, the first non-tumor sample may be a buffy coat sample obtained by a blood draw from the patient. The same buffy coat sample may be saved and used as the second non-tumor sample to screen the preliminary panel. Alternatively, the second non-tumor sample could be a different non-tumor sample, such as a subsequent buffy coat sample obtained from a separate blood draw.

Thus the disclosed methods may comprise obtaining a second non-tumor sample (which may be a saved portion of the first non-tumor sample) from the subject; extracting DNA (e.g., genomic DNA) from the second non-tumor sample; enriching the DNA from the second non-tumor sample for sequences corresponding to the preliminary panel, thereby obtaining an enriched DNA fraction from the second non-tumor sample; sequencing the enriched DNA fraction from the second non-tumor sample to detect the presence or absence of any of the plurality of preliminary tumor-specific somatic mutations; and removing from the preliminary panel any of the plurality of preliminary tumor-specific somatic mutations that are present in the enriched DNA fraction from the second non-tumor sample, thereby obtaining a patient-specific panel.

Additionally, disclosed herein are methods for generating a more accurate patient-specific panel. The present disclosure provides a method of generating a patient-specific panel, including: a) obtaining from a tumor sample and a first non-tumor sample from a subject that has been diagnosed with cancer; b) sequencing DNA from the tumor sample and the first non-tumor sample; c) preparing a preliminary panel including a plurality of preliminary tumor-specific somatic mutations that are present in the tumor sample and absent in the first non-tumor sample; d) obtaining a second non-tumor sample (which may be a saved portion of the first non-tumor sample) from the subject; c) extracting DNA from the second non-tumor sample; f) enriching the DNA from the second non-tumor sample for sequences corresponding to the preliminary panel, thereby obtaining an enriched DNA fraction from the second non-tumor sample; g) sequencing the enriched DNA fraction from the second non-tumor sample to detect the presence or absence of any of the plurality of preliminary tumor-specific somatic mutations; and h) removing from the preliminary panel any of the plurality of preliminary tumor-specific somatic mutations that are present in the enriched DNA fraction from the second non-tumor sample, thereby obtaining a patient-specific panel. The method may further comprise preparing a plurality of oligonucleotide probes, wherein each probe in the plurality of oligonucleotide probes hybridizes to a tumor-specific somatic mutation in the patient-specific signature panel.

The disclosed methods provide two major benefits. First, the additional steps in these methods would be a comprehensive quality control for the bespoke enrichment library before it is applied to a ctDNA sample. Second, these methods enable identification of alterations that are not true somatic changes in the tumor but may be germ-line changes, variants that arise from clonal hematopoiesis of indeterminate potential (CHIP) mutations, sequencing errors, or other artifacts. Removing such artifacts from the patient's panel would lead to a reduction in false positive results and make the test more sensitive for detecting ctDNA and more accurate in quantification of the tumor fraction in cell-free DNA.

The disclosed methods comprise enriching DNA samples. Enriching the DNA from the second non-tumor sample may include contacting the DNA from the second non-tumor sample with a plurality of oligonucleotides, where each oligonucleotide includes a nucleic acid sequence that is capable of hybridizing to a DNA fragment including one of the plurality of preliminary tumor-specific somatic mutations. Enriching the DNA from the second non-tumor sample can include i) hybrid capture-based enrichment, ii) PCR-target enrichment, or iii) on-sequencer enrichment.

The disclosed methods comprise preparing the preliminary panel. Preparing the preliminary panel can include aligning the sequences of DNA from the tumor sample to a reference human genome that is not from the patient, aligning the sequences of DNA from the first non-tumor sample to the reference genome that is not from the patient, and detecting the presence of mutations that are present in the sequences of DNA from the tumor sample but absent in the sequences of DNA from the first non-tumor sample.

The disclosed methods comprise sequencing DNA from the tumor sample and first non-tumor sample. Sequencing DNA may be whole genome sequencing or targeted sequencing. Targeting sequencing may include sequencing of introns, exons, intergenic regions, or a combination thereof.

The disclosed methods comprise preparing a patient-specific panel. The patient-specific panel may include at least 10, at least 50, at least 100, at least 150, at least 200, at least 250, or at least 500 tumor-specific somatic mutations. The patient-specific panel may include one or more somatic mutations selected from SNVs, insertions, deletions, and translocations.

The disclosed methods comprise removing from the preliminary panel any of the plurality of preliminary tumor-specific somatic mutations. The removed preliminary tumor-specific somatic mutations may be selected from germ-line changes, clonal hematopoiesis of indeterminate potential (CHIP) mutations, sequencing errors, and artifacts.

The disclosed methods utilize a first non-tumor sample and a tumor sample to identify tumor-specific and patient specific somatic mutations. The first non-tumor sample may be a tissue sample matched to a tissue of origin of the tumor sample. The first non-tumor sample may be a fluid sample selected from a buffy coat sample, blood, blood plasma, blood serum, urine, saliva, and cerebral spinal fluid (CSF). The method includes a second non-tumor sample. The second non-tumor sample may be a tissue sample matched to a tissue of origin of the tumor sample. The second non-tumor sample may be a fluid sample selected from a buffy coat sample, blood, blood plasma, blood serum, urine, saliva, and cerebral spinal fluid (CSF).

The tumor sample may be a tissue sample, such as a tumor biopsy, or a liquid sample, such as a blood sample (i.e., in the case of blood borne or hematological cancers). The tumor may be selected from adrenal cancer, anal cancer, bile duct cancer, bladder cancer, bone cancer, a brain/CNS tumor, breast cancer, Castleman disease, cervical cancer, colon or rectum cancer, endometrial cancer, esophagus cancer, a Ewing tumor, eye cancer, gallbladder cancer, a gastrointestinal carcinoid tumor, a gastrointestinal stromal tumor (GIST), gestational trophoblastic disease, Hodgkin disease, Kaposi sarcoma, kidney cancer, laryngeal and hypopharyngeal cancer, leukemia, liver cancer, lung cancer, lymphoma, malignant mesothelioma, multiple myeloma, myelodysplastic Syndrome, nasal cavity or paranasal sinus cancer, nasopharyngeal cancer, neuroblastoma, oral cavity or oropharyngeal cancer, osteosarcoma, ovarian cancer, pancreatic cancer, penile cancer, a pituitary tumor, prostate cancer, retinoblastoma, rhabdomyosarcoma, salivary gland cancer, skin cancer, small intestine cancer, stomach cancer, testicular cancer, thymus cancer, thyroid cancer, uterine sarcoma, vaginal cancer, vulvar cancer, Waldenstrom macroglobulinemia, and Wilms tumor. Alternatively, the tumor may be a blood borne or hematological cancer, such as a leukemia or lymphoma.

The subject may be one who has completed at least one cancer treatment prior to obtaining the tumor sample and the non-tumor sample from the subject. The cancer treatment may be selected from chemotherapy, radiotherapy, surgery, immunotherapy, cell therapy, or biologic therapy.

The generated patient-specific panel can be used in methods to detect circulating tumor DNA, to detect minimal residual disease in a subject previously diagnosed with cancer, to monitor treatment in a subject with cancer, and to monitor cancer recurrence or disease progression in a subject previously treated for cancer. Both phases of the MRD assay, as detailed above, can be utilized with the patient specific panels generated via the described methods.

### VI. Methods Utilizing Patient-Specific Panels

Also provided herein are methods utilizing patient-specific panels, as described above. The methods generally include: a) obtaining from a tumor sample and a first non-tumor sample from a subject that has been diagnosed with a cancer; b) sequencing DNA from the tumor sample and the first non-tumor sample; c) preparing a preliminary panel including a plurality of preliminary tumor-specific somatic mutations that are present in the tumor sample and absent in the first non-tumor sample; d) obtaining a second non-tumor sample from the subject; e) extracting DNA from the second non-tumor sample; f) enriching the DNA from the second non-tumor sample for sequences corresponding to the preliminary panel, thereby obtaining an enriched DNA fraction from the second non-tumor sample; g) sequencing the enriched DNA fraction from the second non-tumor sample to detect the presence or absence of any of the plurality of preliminary tumor-specific somatic mutations; h) removing from the preliminary panel any of the plurality of preliminary tumor-specific somatic mutations that are present in the enriched DNA fraction from the second non-tumor sample, thereby obtaining a patient-specific panel; and at one or more later time points: i) obtaining a third non-tumor sample from the subject; j) extracting cfDNA from the third non-tumor sample; k) enriching the cfDNA from the third non-tumor sample for sequences corresponding to the patient-specific panel, thereby obtaining an enriched DNA fraction from the third non-tumor sample; and l) sequencing the enriched DNA fraction from the third non-tumor sample to detect the presence or absence of ctDNA in the third non-tumor sample.

The methods as described involve the generation of patient-specific panels as described previously. The methods may be used to detect minimal residual disease in a subject previously diagnosed with cancer, as presence of ctDNA in the third non-tumor sample is indicative of minimal residual disease in the subject. The methods may be used to monitor treatment in a subject with cancer, as the presence or absence of ctDNA in the third non-tumor sample is indicative of the effectiveness of a treatment. The methods may be used to monitor cancer recurrence or disease progression in a subject previously treated for cancer, as the presence of ctDNA in the third non-tumor sample is indicative of recurrence or progression of disease. The methods may further include providing the subject with a report providing a result to the sequencing of the enriched DNA fraction from the third non-tumor sample.

The method may include a third non-tumor sample. The third non-tumor sample may be a tissue sample matched to a tissue of origin of the tumor sample. The third non-tumor sample may be a fluid sample selected from a buffy coat sample, blood, blood plasma, blood serum, urine, saliva, and cerebral spinal fluid (CSF). The methods may include enriching cfDNA samples. Enriching the cfDNA from the third non-tumor sample can include i) hybrid capture-based enrichment, ii) PCR-target enrichment, or iii) on-sequencer enrichment.

The method may include determining a tumor fraction in the third non-tumor sample. The tumor fraction of zero indicates the absence of the tumor in the subject. The tumor sample may be a solid tumor biopsy or a fluid sample. The fluid sample may be blood, blood plasma, blood serum, urine, saliva, and cerebral spinal fluid (CSF).

The method involves a subject. The subject may be one who has completed at least one cancer treatment prior to obtaining the tumor sample and the non-tumor sample from the subject. The subject may be one who has completed at least one cancer treatment prior to obtaining the third non-tumor sample from the subject. The subject may be one who is receiving at least one cancer treatment at the time of obtaining the third non-tumor sample from the subject. The cancer treatment may be selected from chemotherapy, radiotherapy, surgery, immunotherapy, cell therapy, or biologic therapy.

The method may involve repeating steps (i) through (l) with a fourth, fifth, sixth, seventh, eighth, ninth, or tenth non-tumor sample at successive time points. The method may involve repeating steps (i) through (l) one or more times while the subject is in remission. The method may involve repeating steps (i) through (l) one or more time while the subject is undergoing treatment for the cancer. The method may involve repeating steps (i) through (l) one or more times coinciding with or prior to surgery; following, during, or prior to administration of chemotherapy; following, during, or prior to radiation therapy; following, during, or prior to administration of an immunotherapy; following, during, or prior to administration of a cell therapy; or following, during, or prior to administration of a biologic therapy.

### EXAMPLES

### Methods

A pair of matched tumor and normal cell lines were analyzed by whole genome sequencing to identify somatic variant targets. Oligonucleotide probes targeting those variants were manufactured. The DNA from both the normal and the tumor cell line were sheared to mimic cell-free DNA and mixed at various ratios to generate test samples having tumor DNA concentrations ranging from about 0.005% to 1%. These test samples, along with the tumor and normal control DNA, underwent sequencing library preparation, including addition of unique molecular identifiers (UMIs), hybridization capture using the probe panel, and targeted sequencing. Sequence data was deduplicated using the combined family read approach. Count data of alleles at each target site were gathered, and targets that were detected in the normal control were labeled as “bad” targets. The target data was analyzed using a likelihood model to estimate the tumor fraction of the sample. The analysis was performed many times by applying a resampling approach to select a subset of the total targets to generate a smaller panel. This subsampling approach was applied 100 times for each sample with the following panel sizes (e.g., number of targets): 16, 50, 100, and 150.

### Discussion

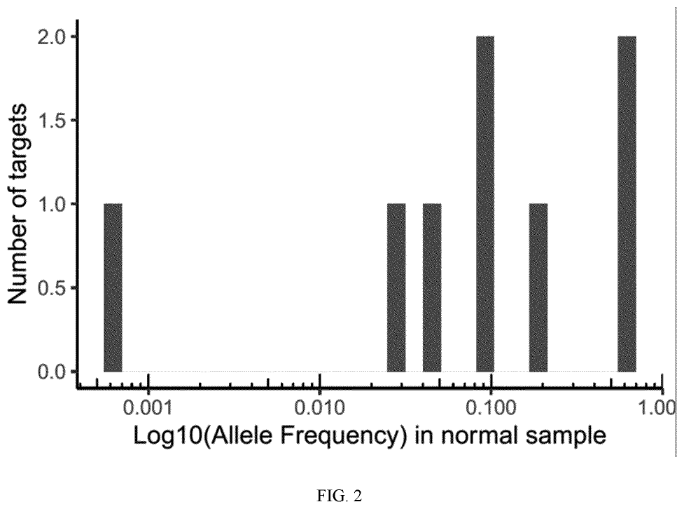

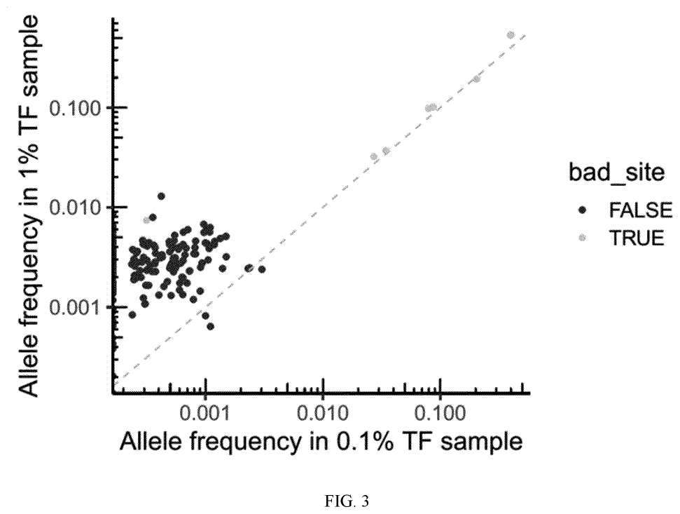

For the pair of matched tumor and normal cell line-derived DNA, eight targets were detected in the normal control at a range of allele frequencies in targeting sequencing (FIG. 2). Because these targets were present in the normal control, their detection did not indicate the presence of tumor DNA. These targets were classified as a false target, meaning that detection of these targets in a plasma sample was not indicative of the presence of tumor DNA. The frequencies of these false targets were often approximately equal regardless of the concentration of tumor DNA in a sample (FIG. 3). Most false targets had similar allele frequencies for each tumor DNA concentration in the generated test samples. This indicated that these false targets were part of the background of all the samples and not associated with tumor DNA concentration. Accordingly, the false targets should be removed from the signature panel prior to further screening/monitoring of a patient.

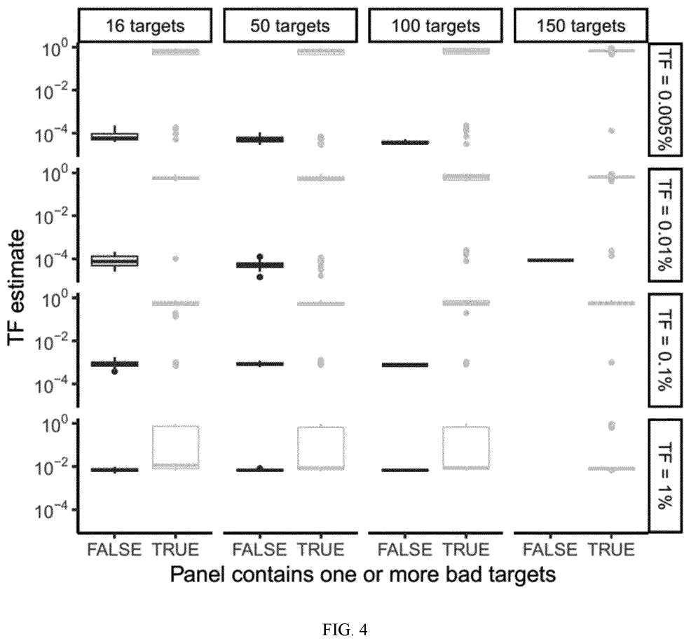

When panels contained these false targets, the estimated tumor DNA fraction was typically greatly overestimated, unless the tumor DNA fraction was relatively high (FIG. 4). Panels with no false targets had lower tumor fraction estimates which was more consistent with the known tumor DNA concentration of each test sample. This was particularly pronounced for the test samples with low tumor DNA concentration. This effect was consistent regardless of panel size; however, larger panels were more likely to include at least one false target.

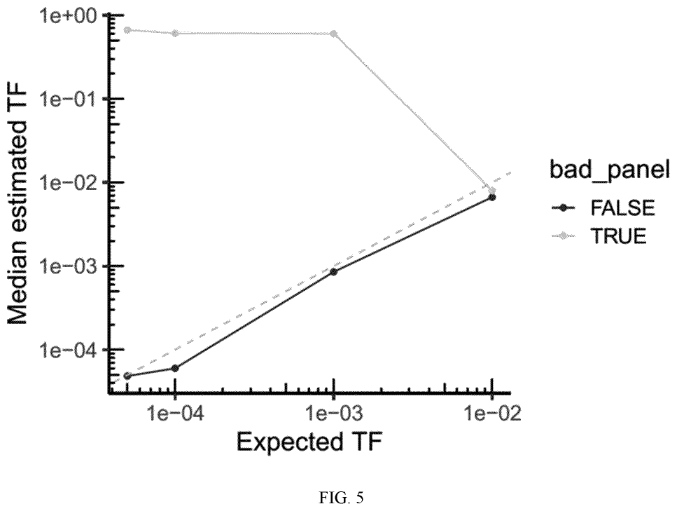

Removal of the false targets from panels resulted in improved accuracy of tumor fraction estimation and specificity (FIG. 5). The median tumor fraction was calculated for each test sample across all simulated panels and compared to the known tumor DNA concentration for the samples. Tumor fraction estimates for panels with false targets were not concordant with the known concentrations.

### Equivalents

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds, compositions or biological systems, which can, of course, vary. It is also to be understood that the terminology used herein is for the purpose of describing particular embodiments only and is not intended to be limiting.

All patents, patent applications, provisional applications, and publications referred to or cited herein are incorporated by reference in their entirety, including all figures and tables, to the extent that are not inconsistent with the explicit teachings of this specification.

What is claimed is:

1. A method of generating a patient-specific panel, comprising:
(a) obtaining from a tumor sample and a first non-tumor sample from a subject that has been diagnosed with a cancer;
(b) sequencing DNA from the tumor sample and the first non-tumor sample;
(c) preparing a preliminary panel comprising a plurality of preliminary tumor-specific somatic mutations that are present in the tumor sample and absent in the first non-tumor sample;
(d) obtaining a second non-tumor sample from the subject;
(e) extracting DNA from the second non-tumor sample;
(f) enriching the DNA from the second non-tumor sample for sequences corresponding to the preliminary panel, thereby obtaining an enriched DNA fraction from the second non-tumor sample;
(g) sequencing the enriched DNA fraction from the second non-tumor sample to detect the presence or absence of any of the plurality of preliminary tumor-specific somatic mutations; and
(h) removing from the preliminary panel any of the plurality of preliminary tumor-specific somatic mutations that are present in the enriched DNA fraction from the second non-tumor sample, thereby generating a patient-specific panel.

2. The method of claim 1 further comprising preparing a plurality of oligonucleotide probes, wherein each probe in the plurality of oligonucleotide probes hybridizes to a tumor-specific somatic mutation in the patient-specific signature panel.

3. A method of detecting circulating tumor DNA (ctDNA) in a sample, comprising:
(a) obtaining from a tumor sample and a first non-tumor sample from a subject that has been diagnosed with a cancer;
(b) sequencing DNA from the tumor sample and the first non-tumor sample;
(c) preparing a preliminary panel comprising a plurality of preliminary tumor-specific somatic mutations that are present in the tumor sample and absent in the first non-tumor sample;
(d) obtaining a second non-tumor sample from the subject;
(e) extracting DNA from the second non-tumor sample;
(f) enriching the DNA from the second non-tumor sample for sequences corresponding to the preliminary panel, thereby obtaining an enriched DNA fraction from the second non-tumor sample;
(g) sequencing the enriched DNA fraction from the second non-tumor sample to detect the presence or absence of any of the plurality of preliminary tumor-specific somatic mutations;
(h) removing from the preliminary panel any of the plurality of preliminary tumor-specific somatic mutations that are present in the enriched DNA fraction from the second non-tumor sample, thereby obtaining a patient-specific panel; and
at one or more later time points:
(i) obtaining a third non-tumor sample from the subject;
(j) extracting cell-free DNA (cfDNA) from the third non-tumor sample;
(k) enriching the cfDNA from the third non-tumor sample for sequences corresponding to the patient-specific panel, thereby obtaining an enriched DNA fraction from the third non-tumor sample; and
(l) sequencing the enriched DNA fraction from the third non-tumor sample to detect the presence or absence of ctDNA in the third non-tumor sample.

4. The method of claim 3, where the first non-tumor sample and the second non-tumor sample are the same sample.

5. The method of claim 3, wherein enriching the cfDNA from the second non-tumor sample comprises (i) hybrid capture-based enrichment, (ii) PCR-target enrichment, or (iii) on-sequencer enrichment.

6. The method of claim 3, wherein enriching the cfDNA from the third non-tumor sample comprises (i) hybrid capture-based enrichment, (ii) PCR-target enrichment, or (iii) on-sequencer enrichment.

7. The method of claim 3 further comprising providing the subject with a report providing a result to the sequencing of the enriched DNA fraction from the third non-tumor sample.

8. The method of claim 3, wherein sequencing DNA from the tumor sample and the first non-tumor sample comprises whole genome sequencing.

9. The method of claim 3, wherein sequencing DNA from the tumor sample and the first non-tumor sample comprises targeted sequencing.

10. The method of claim 3, wherein the patient-specific panel comprises at least 10 tumor-specific somatic mutations.

11. The method of claim 3, wherein any of the plurality of preliminary tumor-specific somatic mutations that are removed from the preliminary panel are alterations selected from germ-line changes, clonal hematopoiesis of indeterminate potential (CHIP) mutations, sequencing errors, and artifacts.

12. The method of claim 3, wherein the tumor sample comprises a solid tumor biopsy or a fluid sample.

13. The method of claim 3, wherein the first non-tumor sample comprises a fluid sample selected from a buffy coat sample, blood, blood plasma, blood serum, urine, saliva, and cerebral spinal fluid (CSF).

14. The method of claim 3, wherein the second non-tumor sample comprises a fluid sample selected from a buffy coat sample, blood, blood plasma, blood serum, urine, saliva, and cerebral spinal fluid (CSF).

15. The method of claim 3, wherein the third non-tumor sample comprises a fluid sample selected from a buffy coat sample, blood, blood plasma, blood serum, urine, saliva, and cerebral spinal fluid (CSF).

16. The method of claim 3, wherein the subject has completed at least one cancer treatment prior to obtaining the tumor sample and the first and second non-tumor sample from the subject.

17. The method of claim 3, wherein the subject has completed at least one cancer treatment prior to obtaining the third non-tumor sample from the subject.

18. The method of claim 3, wherein the subject is receiving at least one cancer treatment at the time of obtaining the third non-tumor sample from the subject.

19. The method of claim 3, wherein the tumor is selected from adrenal cancer, anal cancer, bile duct cancer, bladder cancer, bone cancer, a brain/CNS tumor, breast cancer, Castleman disease, cervical cancer, colon or rectum cancer, endometrial cancer, esophagus cancer, a Ewing tumor, eye cancer, gallbladder cancer, a gastrointestinal carcinoid tumor, a gastrointestinal stromal tumor (GIST), gestational trophoblastic disease, Hodgkin disease, Kaposi sarcoma, kidney cancer, laryngeal and hypopharyngeal cancer, leukemia, liver cancer, lung cancer, lymphoma, malignant mesothelioma, multiple myeloma, myelodysplastic Syndrome, nasal cavity or paranasal sinus cancer, nasopharyngeal cancer, neuroblastoma, oral cavity or oropharyngeal cancer, osteosarcoma, ovarian cancer, pancreatic cancer, penile cancer, a pituitary tumor, prostate cancer, retinoblastoma, rhabdomyosarcoma, salivary gland cancer, skin cancer, small intestine cancer, stomach cancer, testicular cancer, thymus cancer, thyroid cancer, uterine sarcoma, vaginal cancer, vulvar cancer, Waldenstrom macroglobulinemia, and Wilms tumor.

20. A method of detecting minimal residual disease in a subject previously diagnosed with cancer, comprising:
(a) obtaining from a tumor sample and a first non-tumor sample from the subject;
(b) sequencing DNA from the tumor sample and the first non-tumor sample;
(c) preparing a preliminary panel comprising a plurality of preliminary tumor-specific somatic mutations that are present in the tumor sample and absent in the first non-tumor sample;
(d) obtaining a second non-tumor sample from the subject;
(e) extracting DNA from the second non-tumor sample;
(f) enriching the DNA from the second non-tumor sample for sequences corresponding to the preliminary panel, thereby obtaining an enriched DNA fraction from the second non-tumor sample;
(g) sequencing the enriched DNA fraction from the second non-tumor sample to detect the presence or absence of any of the plurality of preliminary tumor-specific somatic mutations;
(h) removing from the preliminary panel any of the plurality of preliminary tumor-specific somatic mutations that are present in the enriched DNA fraction from the second non-tumor sample, thereby obtaining a patient-specific panel; and
at one or more later time points:
(i) obtaining a third non-tumor sample from the subject;
(j) extracting cfDNA from the third non-tumor sample;
(k) enriching the cfDNA from the third non-tumor sample for sequences corresponding to the patient-specific panel, thereby obtaining an enriched DNA fraction from the third non-tumor sample; and
(l) sequencing the enriched DNA fraction from the third non-tumor sample to detect the presence or absence of ctDNA in the third non-tumor sample, wherein the presence of ctDNA in the third non-tumor sample is indicative of minimal residual disease in the subject.
